(19) United States
(12) Patent Application Publication
(10) Pub. No.: US 2025/0230318 A1
(43) Pub. Date: Jul. 17, 2025

(54) REAGENTS FOR FLUORESCENT, UV, AND MS LABELING OF O-GLYCANS AND METHODS OF MAKING AND USING THEM

(71) Applicant: Agilent Technologies, Inc., Santa Clara, CA (US)

(72) Inventors: Ace Gita GALERMO, Greenfield, CA (US); Li-Chih HU, San Jose, CA (US)

(73) Assignee: Agilent Technologies, Inc., Santa Clara, CA (US)

(21) Appl. No.: 18/730,777

(22) PCT Filed: Feb. 26, 2023
(86) PCT No.: PCT/US2023/013911

§ 371 Date: Jul. 21, 2024

Related U.S. Application Data
(60) Provisional application No. 63/403,828, filed on Sep. 5, 2022.
(60) Provisional application No. 63/314,398, filed on Feb. 26, 2022.

(51) Int. Cl. C09B 57/02; G01N 30/02; G01N 30/72; G01N 30/88; G01N 33/533; G01N 33/68; G01N 33/74

(52) U.S. Cl. CPC C09B 57/02; G01N 30/72; G01N 30/88; G01N 33/533; G01N 33/6848; G01N 33/74; G01N 2030/027; G01N 2030/8831; G01N 2030/8836; G01N 2400/10

(57) ABSTRACT

The present disclosure provides new reagents for labeling and detecting O-glycans released from a glycoconjugate, such as a glycoprotein, glycopeptide, peptidoglycan, or proteoglycan of interest. O-glycans labeled with the labels can be detected by fluorescence, MS, and UV. The invention further provides methods for making the reagents, methods for using them, and kits comprising them. O-glycans labeled with the reagents can be analyzed by high-throughput analysis.

### CROSS-REFERENCE TO RELATED APPLICATIONS

This application claims priority to, and the benefit of, U.S. Provisional Patent Application No. 63/314,398, filed Feb. 26, 2022, and U.S. Provisional Patent Application No. 63/403,828, filed Sep. 5, 2022. The contents of both of these applications are incorporated herein by reference.

### STATEMENT OF FEDERAL FUNDING

Not applicable.

### BACKGROUND OF THE INVENTION

This invention relates to the field of labeling O-glycans with a label that allows for detection by fluorescence, by mass spectrometry, and by ultra-violet absorption.

Many of the proteins or peptides produced by eukaryotic cells are modified after translation by the addition of moieties such as lipids or carbohydrates, or by phosphorylation. These modifications can significantly affect the properties of the modified protein or peptide.

One important group of such post-translational modification is the addition of covalently-linked, linear or branched chains of carbohydrates. Protein-carbohydrate conjugates are referred to as glycoproteins, while peptides with attached carbohydrates are referred to as glycopeptides. The point at which the carbohydrate is attached to the protein or peptide is referred to as a glycosylation site. Attached polysaccharides and oligosaccharides are sometimes referred to herein as glycans. N-glycans are defined as those attached to the protein or peptide through the nitrogen of an asparagine residue, while O-glycans are defined as those attached to the protein or peptide through the hydroxyl oxygen of serine, threonine, tyrosine, hydroxylysine, or hydroxyproline side-chains.

The particular pattern of glycans on a particular glycoprotein or glycopeptide is determined by the specific cell line that produced the protein or peptide and the conditions under which the cells were grown. Since the glycans conjugated to a protein or peptide can affect characteristics critical to its function, including pharmacokinetics, stability, bioactivity, or immunogenicity, in many uses it is important to determine which glycans are present. For example, the Food and Drug Administration requires characterization of carbohydrates attached to therapeutic glycoproteins and vaccines to show composition of matter and consistency of manufacture, resulting in a need for extensive characterization of the product. Analysis of the profile of the carbohydrates is also important for quality control in the production of recombinant proteins or peptides, in which a change in carbohydrate profile may indicate stress in the system, which if not corrected in time may require a commercial-scale fermenter of expensive protein to be discarded.

Current methods for determining which glycans are present on a glycoprotein or glycopeptide of interest typically rely on releasing the glycans from the protein component, a process referred to as “deglycosylation.” Many N-glycans can conveniently be released from glycoproteins or glycopeptides under mild conditions by enzymatic cleavage by various enzymes, such as PNGase F (Peptide-N4-(acetyl-β-glucosaminyl)-asparagine amidase, EC 3.5.1.52). Enzymatic digestion of N-glycans, such as by PNGase F, typically occurs in an aqueous solution, and results in the initial release of the N-glycans as β-glycosylamines, in which the free-reducing end of the released glycan is conjugated with ammonia (see, e.g., Tarentino, et al. TIGG 1993, 23, 163-170; Rasmussen J. R. J. Am. Chem. Soc. 1992, 114, 1124-1126; Risley, et al., J. Biol. Chem. 1985, 260, 15488-15494, 1985).

Unfortunately, only limited means of releasing O-glycans by enzymes are available. The disaccharide Gal-β(1,3) GalNAc, can be released enzymatically by the enzyme O-Glycosidase, but, in general, there are no enzymatic means of releasing intact O-glycans with branching chains. As a result, a number of methods have been developed which release these more complex O-glycans by use of chemicals. Once the O-glycans have been released from the glycoprotein or glycopeptide, they are typically labeled at their reducing terminus, subjected to high pressure liquid chromatography to separate the labeled glycans, and analyzed to determine the types and amounts of glycans released from the glycoprotein or glycopeptide of interest.

Most of the traditional chemical deglycosylation techniques involve chemical deglycosylation of a relatively high amount of starting sample under relatively harsh conditions. A relatively early protocol for releasing O-glycans, for example, called for incubation with trifluoro-methanesulfonic acid at 0° C. for 0.5 to 2 hours, followed by the neutralization of the acid with aqueous pyridine at −20° C. The Carlson method, which many consider the gold standard for releasing and analyzing O-glycans, dates from the late 1960s, and relies on beta-elimination, that is, the release of O-glycans under basic conditions, using sodium hydroxide in combination with reduction using sodium borohydride, incubation at 45° C. for 14-16 hours, and solid-phase extraction to enrich the glycans for analysis by liquid chromatography-mass spectrometry (“LC-MS”). See, e.g., Carlson, D., J. Biol. Chem., 243:616-626 (1968). The Maniatis method requires 1+ milligram of glycoprotein and relies on beta-elimination using dimethylamine, microwave radiation at 70° C. for 70 minutes, and permethylation of hydroxyl groups for analysis by matrix-assisted laser desorption ionization mass spectrometry (MALDI-MS). See, e.g., Maniatis, et al., Anal. Chem. 82:2421-2425 (2010). The Merry et al. method uses hydrazine, a toxic and unstable compound when in anhydrous form, requires >40 μg of glycoprotein, causes unwanted removal of acetyl groups that may be present in the O-glycan core, incubation at 60° C. for 6 hours, and additional steps of re-acetylation and reducing-end labeling with a fluorophore. See, e.g., Merry, et al., Anal. Biochem. 304:91-99 (2002). Others reported using releasing O-glycans using a combination beta-elimination, 50% hydrazine solution with 0.2M triethylamine, followed by labeling with para-amino benzooic acid ethyl ester, and found good results with an optimal incubation time of 48 hours. See, Kisiel et al., Toxicology Mechanisms and Methods, 18:503-07 (2008); Kisiel, et al., Acta Biochemica Polonica, 1999, 46(3):753-757.

More recently, several “one-pot” procedures have been developed. One such one-pot approach uses beta-elimination with dimethylamine in combination with reducing-end labeling with 1-phenyl-3-methyl-5-pyrazolone, or “PMP”. The method calls for the incubation of a very small sample, on the order of 5 μg, with 40% dimethylamine in 0.5 M PMP in methanol at 85° C. for 2 hours, followed by a dry-down of sample and solid-phase extraction of the released glycans to concentrate them for analysis for liquid chromatography-mass spectrometry (LC-MS). See, e.g, Zauner, et al., Biochim. Biophys. Acto. 1820:1420-1428 (2012) (hereafter, “Zauner”). Wang, et al. (Proteomics 11:4229-4242 (2011)) and Furukawa, et al. (Anal. Chem, 83:9060-9067 (2011)) are other one-pot beta elimination approaches for releasing O-glycans and labeling them with PMP. Wuhrer et al. teach what they assert is “a one-pot process for releasing and labeling O-glycans from glycoproteins . . . comprising contacting the glycoprotein with an amine, eg, methylamine, dimethylamine, or ammonia, NH3 in the presence of a labeling agent.” Abstract, Wuhrer, et al., WO 2011/038874. Most recently, a one-pot procedure using PMP as a label was disclosed in a co-owned PCT application, published as WO 2021/119333.

N-glycans released from glycoproteins or glycopeptides are typically labeled with labels, such as InstantPC® (Agilent Technologies, Inc., Santa Clara, CA), that are detectable by both fluorescence and by mass spectrometry (“MS”). This allows the N-glycans to be quantitated by detecting their fluorescent signal, and their molecular species to then be determined by MS analysis. Unfortunately, no single available label for O-glycans combines all the features that would be desirable. While the label 2-aminobenzamide (“2-AB”) provides O-glycans with a label that is detectable by both fluorescence and MS, it cannot be used in one-pot release and labeling methods. And, while PMP can be used in such one-pot processes, and provides O-glycans with a signal detectable by MS, it is not fluorescent. It would be desirable to have a reagent that could be used in one-pot methods to release and label O-glycans and that would provide the resulting labeled O-glycan derivatives with a label that can be detected by UV, by fluorescence, and by MS.

There remains a need for reagents that can be used to label O-glycans released from glycoproteins or glycopeptides so they can be detected by UV, fluorescence, and MS, that can be used in a one-pot procedure, that require only small sample volumes, that require only moderate reaction conditions, that require only short incubation times, and that can be adapted for high-throughput analysis. Surprisingly, the present invention fulfills these and other needs.

### BRIEF SUMMARY OF INVENTION

In a first group of embodiments, the invention provides fluorescent O-glycan labeling compounds, comprising a pyrazolone core, a linker, and, a coumarin core, wherein, (a) said linker covalently links said pyrazolone core to said coumarin core, and (b) when said pyrazolone core is reacted with an O-glycan to label said O-glycan, thereby forming an O-glycan labeled with a derivative of said O-glycan labeling compound, said O-glycan labeled with said derivative of said O-glycan labeling compound is detectable by fluorescence. In some embodiments, the linker covalently linking said pyrazolone core to said coumarin core is comprised of (a) an alkyl chain of ten or fewer carbon atoms, and (b) a chain of up to ten carbon atoms, or of a total of ten carbon atoms and bridging groups, provided that there is at least one carbon atom between said pyrazolone core and any bridging groups, provided that at least one unsubstituted carbon atom is disposed between any two bridging groups. In some embodiments, the bridging groups are selected from an ester group, an amine group, an alkyl group, a benzyl group, and a thioester group. In some embodiments, the linker covalently linking said pyrazolone core to said coumarin core is comprised of four or fewer carbon atoms. In some embodiments, the linker is one carbon atom. In some embodiments, the pyrazolone is covalently linked to said coumarin core through a linker of a chain, in order from said pyrazolone core to said coumarin core, of a first carbon atom, a second carbon atom, and an oxygen atom in an ether linkage from said second carbon atom to an atom of said coumarin core. In some embodiments, the pyrazolone is covalently linked to said coumarin core through a linker of a chain, in order from said pyrazolone core to said coumarin core, of a first carbon atom, a second carbon atom, and a nitrogen atom in an amide linkage from said second carbon atom to an atom of said coumarin core, which nitrogen atom can have a substituent of a hydrogen or an alkyl group having the formula CₙH₂ₙ₊₁. In some embodiments, the pyrazolone is covalently linked to said coumarin core through a linker of a chain of up to nine carbon atoms and a benzyl group. In some embodiments, the pyrazolone is covalently linked to said coumarin core through a linker, in order from said pyrazolone core, of one carbon atom and a benzyl group. In some embodiments, the pyrazolone is covalently linked to said coumarin core through a linker of a chain of carbon atoms and carbon atoms bearing a carbonyl group, provided that at least one unsubstituted carbon atom is disposed between each carbon atom bearing a carbonyl group. In some embodiments, the compound has the structure of:
Structure 3,

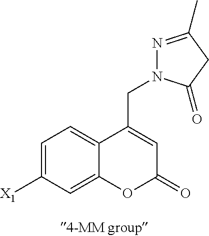

wherein X₁=Cl, Br, I, OH, OMe, OEt, NH₂, NMe₂, NEt₂,
of Structure 4

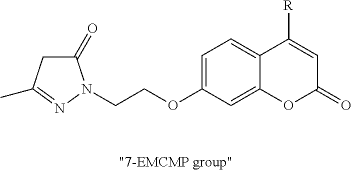

wherein R=CH₃ or CF₃,
of Structure 5,

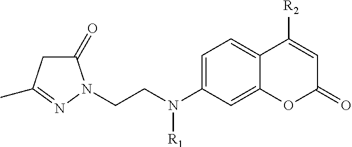

wherein R₁=H or CₙH₂ₙ₊₁; and R₂=CH₃ or CF₃,
of Structure 6,

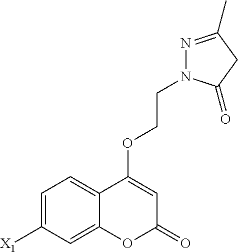

wherein X₁=H, Cl, Br, I, OH, OMe, OEt, NH₂, NMe₂, NEt₂,
of Structure 7,

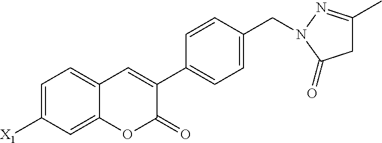

wherein X₁=H, Cl, Br, I, OH, OMe, OEt, NH₂, NMe₂, NEt₂, or
of Structure 8,

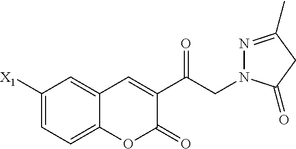

wherein X₁=H, Cl, Br or I.

In some embodiments, the, compound is a compound of Structure 3, and has the structure of Structure 1:

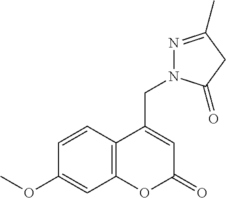 [SMILES: COc1ccc2c(CN3N=C(C)CC3=O)cc(=O)oc2c1]

(“4-MM”). In some embodiments, the compound is a compound of Structure 4, and has the structure:

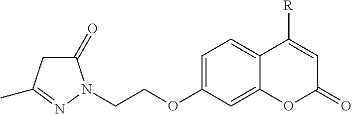

wherein R=CH₃ (“7-EMCMP”).

In another group of embodiments, the invention provides methods for releasing, labeling, and, optionally, analyzing, O-glycans present on a selected glycoconjugate of interest, said method comprising: incubating in a container said selected glycoconjugate of interest in a solution comprising a fluorescent O-glycan labeling compound, said compound comprising a pyrazolone core, a linker, and, a coumarin core, wherein (a) said linker covalently links said pyrazolone core to said coumarin core, and (b) when said pyrazolone core is reacted with an O-glycan to label said O-glycan, thereby forming an O-glycan labeled with a derivative of said O-glycan labeling compound, said O-glycan labeled with said derivative of said O-glycan labeling compound is detectable by fluorescence, at a temperature of 0° C. to 100° C. for a time between about 10 minutes and 23 hours. In some embodiments, the linker covalently linking said pyrazolone core to said coumarin core is comprised of: (a) an alkyl chain of ten or fewer carbon atoms, (b) a chain of up to ten carbon atoms, or of a total of ten carbon atoms and bridging groups, provided that there is at least one carbon atom between said pyrazolone core and any bridging groups, provided that at least one unsubstituted carbon atom is disposed between any two bridging groups. In some embodiments, the bridging group or groups is/are independently selected from an ester group, an amine group, an alkyl group, a benzyl group, and a thioester group. In some embodiments, the fluorescent O-glycan labeling compound has the structure:
of Structure 3,

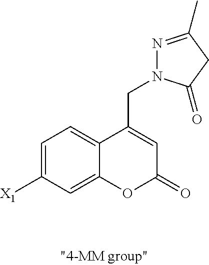

wherein X₁=Cl, Br, I, OH, OMe, OEt, NH₂, NMe₂, NEt₂,
of Structure 4

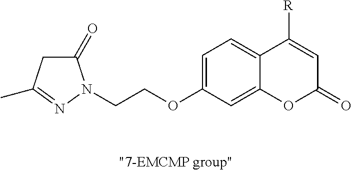

wherein R=CH₃ or CF₃,
of Structure 5,

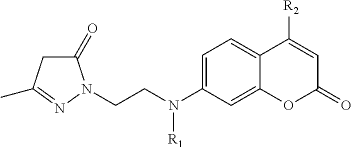

wherein R₁=H or CₙH₂ₙ₊₁; R₂=CH₃ or CF₃,
of Structure 6,

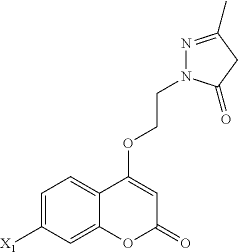

wherein X₁=H, Cl, Br, I, OH, OMe, OEt, NH₂, NMe₂, NEt₂,
of Structure 7

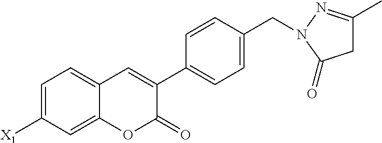

wherein X₁=H, Cl, Br, I, OH, OMe, OEt, NH₂, NMe₂, NEt₂, or
of Structure 8,

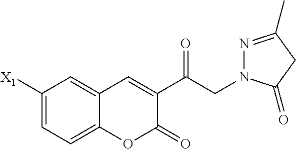

X₁=H, Cl, Br or I. In some embodiments, the fluorescent O-glycan labeling compound is a compound of Structure 3, and has the structure of Structure 1:

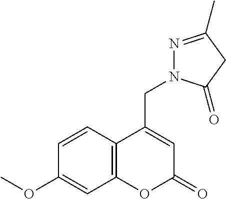 [SMILES: COc1ccc2c(CN3N=C(C)CC3=O)cc(=O)oc2c1]

(“4-MM”). In some embodiments, the fluorescent O-glycan labeling compound is a compound of Structure 4, and has the structure:

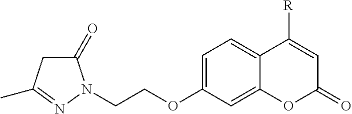

wherein R=CH₃ (“7-EMCMP”). In some embodiments, the linker of said fluorescent O-glycan labeling compound comprises a total of four or fewer carbons and bridging groups, and the solution is wholly aqueous. In some embodiments, the linker of said fluorescent O-glycan labeling compound comprises a total of four or fewer carbons and bridging groups and the solution in a mixture of aqueous solution and an organic solvent that is either a polar protic organic solvent or an aprotic organic solvent. In some embodiments, the organic solvent is a polar protic organic solvent. In some embodiments, the polar protic organic solvent is methanol. In some embodiments, the linker of said fluorescent O-glycan labeling compound comprises a total of five to ten carbons and bridging groups and the solution comprises up to 90% polar protic or aprotic organic solvent. In some embodiments, the solution further comprises an effective amount of a base. In some embodiments, the mixture of aqueous solution and organic solvent further comprises an effective amount of a base. In some embodiments, the solution comprising up to 90% polar protic or aprotic organic solvent further comprises an effective amount of a base. In some embodiments, the base is dimethylamine, triethylamine (“TEA”), sodium hydroxide, potassium hydroxide, lithium hydroxide, ammonium hydroxide, or a combination of two or more of these. In some embodiments, base is TEA. In some embodiments, base is present at a concentration of 0.5M to 5M. In some embodiments, the base is TEA and is present at a concentration of 0.5M to 5M. In some embodiments, the fluorescent O-glycan labeling compound is present at a concentration of 0.05M to 5M. In some embodiments, the glycoconjugate is incubated in said aqueous solution at a temperature of 0-100° C. for a time between about 10 minutes and 23 hours. In some embodiments, the selected glycoconjugate of interest is present in said solution in an amount between 4 μg and 1000 μg. In some embodiments, the selected glycoconjugate of interest is present in said solution in an amount between 20 μg and 800 μg. In some embodiments, the selected glycoconjugate of interest is a glycoprotein or a glycopeptide. In some embodiments, the aqueous solution contains no hydrazine. In some embodiments, the time of incubation is from 10 to about 30 minutes. In some embodiments, the temperature at which said aqueous solution is incubated is 70-90° C. In some embodiments, the temperature at which said aqueous solution is incubated is 80° C.±5° C. In some embodiments, the container is a multi-well plate. In some embodiments, the multi-well plate is a 96-well plate. In some embodiments, the methods further comprise analyzing said released, labeled O-glycans. In some embodiments, the analysis comprises separating said released, labeled O-glycans by liquid chromatography. In some embodiments, the analysis comprises analyzing said released, labeled, separated O-glycans by fluorescence. In some embodiments, the analysis comprises analyzing said released, labeled, separated O-glycans by mass spectrometry. In some embodiments, the analysis comprises separating said released, labeled O-glycans by liquid chromatography and analyzing said released, labeled, and separated O-glycans by fluorescence, ultraviolet light, mass spectrometry, or a combination of two or more of these.

In yet a further group of embodiments, the invention provides compositions comprising a glycoconjugate of interest and a solution comprising (a) a base selected from dimethylamine, triethylamine (“TEA”), sodium hydroxide, potassium hydroxide, lithium hydroxide, and ammonium hydroxide, and (b) a fluorescent O-glycan labeling compound, said compound comprising a pyrazolone core, a linker, and, a coumarin core, wherein (i) said linker covalently links said pyrazolone core to said coumarin core, and (ii) when said pyrazolone core is reacted with an O-glycan to label said O-glycan, thereby forming an O-glycan labeled with a derivative of said O-glycan labeling compound, said O-glycan labeled with said derivative of said O-glycan labeling compound is detectable by fluorescence. In some embodiments, the fluorescent O-glycan labeling compound has the structure
of Structure 3,

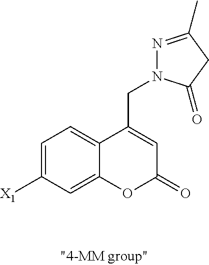

wherein X₁=Cl, Br, I, OH, OMe, OEt, NH₂, NMe₂, NEt₂,
   of Structure 4,

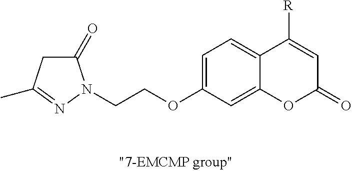

wherein R=CH₃ or CF₃,
of Structure 5,

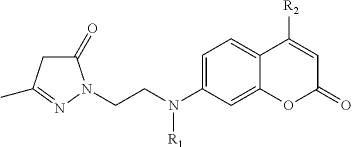

wherein R₁=H or CₙH₂ₙ₊₁; and R₂=CH₃ or CF₃,
of Structure 6,

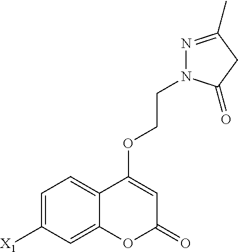

wherein X₁=H, Cl, Br, I, OH, OMe, OEt, NH₂, NMe₂, NEt₂,
of Structure 7,

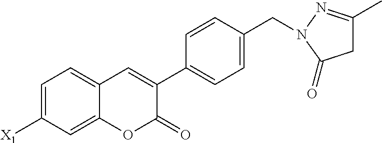

wherein X₁=H, Cl, Br, I, OH, OMe, OEt, NH₂, NMe₂, NEt₂, or
of Structure 8,

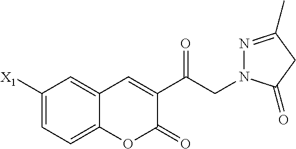

wherein X₁=H, Cl, Br or I. In some embodiments, the fluorescent O-glycan labeling compound is a compound of Structure 3, and has the structure of Structure 1:

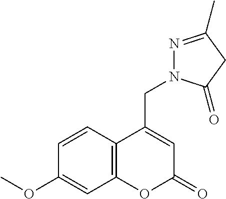 [SMILES: COc1ccc2c(CN3N=C(C)CC3=O)cc(=O)oc2c1]

(“4-MM”). In some embodiments, the fluorescent O-glycan labeling compound is a compound of Structure 4, and has the structure:

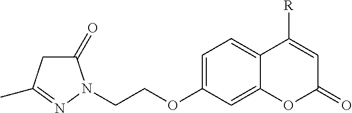

wherein R=CH₃ (“7-EMCMP”). In some embodiments, the solution contains no more than 5% hydrazine. In some embodiments, the solution contains no hydrazine.

In another group of embodiments, the invention provides methods of synthesizing 1-(4-methyl-7-methoxycoumarin)-3-methyl-5-pyrazolone (“4-MM”), said methods comprising:
(a) reacting 4-bromomethyl-7-methoxycoumarin and t-butyl carbazate in the presence of a water soluble carbonate or bicarbonate, in a polar aprotic organic solvent at a temperature and for a time sufficient to result in Reaction Product 1,
(b) removing said solvent, thereby obtaining a crude solid mixture of Reaction Product 1, (c) adding an acid selected from trifluoracetic acid (“TFA”), glacial acetic acid, glacial formic acid, trichloroacetic acid to said crude solid mixture of Reaction Product 1, and allowing said acid to react with said crude solid mixture of Reaction Product 1 at a temperature and for a time sufficient to result in the formation of Reaction Product 2,
(d) adding an acetoacetic hydrocarbon ester to said Reaction Product 2 and incubating the resulting mixture for a time and at a temperature allowing it to form Reaction Product 3 in a solution having a pH, (e) adjusting said pH of said solution to between pH 3-7, and,
(f) extracting said Reaction Product 3 (4-MM) from said solution, thereby obtaining 4-MM. In some embodiments, the temperature in step (a) is ≥40° C.≤90° C. In some embodiments, the temperature in step (a) is 65° C.±10° C. In some embodiments, the time in step (a) is 16 hours, ±4 hours. In some embodiments, the removal of said solvent is by rotavap. In some embodiments, the water soluble carbonate or bicarbonate is sodium carbonate or sodium bicarbonate. In some embodiments, the polar aprotic organic solvent is tetrahydrofuran, acetone, or dimethylformamide. In some embodiments, the acid is TFA. In some embodiments, excess t-butyl carbazate is removed between steps (a) and (c) by extracting it from said crude solid mixture with dichloromethane, diethyl ether or ethyl acetate, against saturated sodium bicarbonate. In some embodiments, the time in step (c) is 1 hour, ±0.25 hours. In some embodiments, the temperature in step (c) is room temperature, ±5° C. In some embodiments, the time of said incubation in step (d) is 1-24 hours. In some embodiments, the time of said incubation in step (d) is 3-16 hours. In some embodiments, the temperature of said incubation in step (d) is room temperature, ±5° C. In some embodiments, the adjusting of said pH in step (e) is by adding sodium acetate, Na₂CO₃, NaHCO₃, NH₄Cl, NaOH, HCl or TFA, or a combination of any of these. In some embodiments, the acetoacetic hydrocarbon ester is selected from the group consisting of acetoacetic ethyl ester, acetoacetic methyl ester, acetoacetic propyl ester, acetoacetic butyl ester, acetoacetic pentyl ester, acetoacetate benzyl ester, or acetoacetate phenyl ester.

In another group of embodiments, the invention provides an additional set of methods for synthesizing 1-(4-methyl-7-methoxycoumarin)-3-methyl-5-pyrazolone (“4-MM”), said methods comprising: (a) reacting 4-bromomethyl-7-methoxycoumarin and t-butyl carbazate in the presence of a water soluble bicarbonate or carbonate, in a polar aprotic organic solvent at a temperature and for a time sufficient to result in Reaction Product 1,
(b) removing said solvent, thereby obtaining a crude solid mixture of Reaction Product 1,
(c) adding an alcohol to said crude solid mixture of Reaction Product 1, and heating the resulting mixture at a temperature and for a time sufficient to dissolve said solids in said crude solid mixture, (d) adding an acid to said mixture containing said dissolved and incubating the resulting mixture for a time and at a temperature to allow the formation of solids in said mixture, (e) filtering said solids to separate them from said mixture, and incubating said separated solids with an alcohol and an acetoacetic hydrocarbon ester for a time and at a temperature sufficient to allow a reaction between said separated solids and said acetoacetic hydrocarbon ester to form 4-MM, (f) extracting said mixture with a suitable organic solvent to concentrate 4-MM in an organic layer and purifying said 4-MM from said organic layer, thereby synthesizing 4-MM. In some embodiments, the methods further comprise isolating 4-MM from said purified 4-MM. In some embodiments, the polar aprotic organic solvent in step (a) is tetrahydrofuran, acetone, or dimethylformamide. In some embodiments, the removal of said solvent in step (b) is by rotavap. In some embodiments, the temperature in step (a) is 75° C.±10° C. In some embodiments, the time in step (a) is 6 hours, ±3 hours. In some embodiments, the temperature in step (c) is 50° C.±10° C. In some embodiments, the time in step (d) is 30 minutes, ±10 hours. In some embodiments, the temperature in step (e) is 80° C.±10° C. In some embodiments, the time in step (e) is 16 hours, ±6 hours. In some embodiments, the acetoacetic hydrocarbon ester added in step (e) is at a concentration of 20 mmol±10 mmol. In some embodiments, the acetoacetic hydrocarbon ester added in step (e) is at a concentration of 20 mmol±5 mmol. In some embodiments, the organic solvent of step (f) is ethyl acetate, dichloro methane, or dimethyl ether. In some embodiments, the isolation is by filtration. In some embodiments, the isolation is by rotary evaporation. In some embodiments, the acetoacetic hydrocarbon ester of step (e) is selected from the group consisting of acetoacetic ethyl ester, acetoacetic methyl ester, acetoacetic propyl ester, acetoacetic butyl ester, acetoacetic pentyl ester, acetoacetate benzyl ester, or acetoacetate phenyl ester.

In still another group of embodiments, the invention provides methods of synthesizing the compound 1-[7-ethoxy(-4-methylcoumarin)]-3-methyl-5-pyrazolone (“7-EMCMP”), which compound has the structure of structure 4,

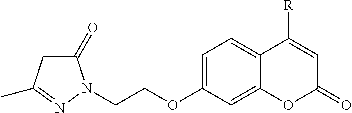

wherein R=CH₃,
said method comprising the following steps in the following order:
(a) reacting 4-methylumbelliferone and MeONa in MeOH in a reaction vessel to obtain a yellow solution, (b) adding dimethylformamide (DMF), followed by 2-chloroethyl p-toluenesulfonate under stirring and purging with nitrogen, thereby creating a first mixture, (c) slowly heating the first mixture of step (b) to a first selected temperature above room temperature, incubating the first mixture at or about the selected temperature for an effective period of time, cooling the first mixture to a second selected temperature above room temperature, adding an alcohol selected from ethanol, methanol, propanol, butanol, thereby creating a second mixture, and heating the second mixture to boiling, cooling the second mixture to room temperature, collecting any solids in said reaction vessel, and washing said solids with cold ethanol, methanol, propanol, or butanol, (d) placing the washed solids of step (c) in a reaction vessel, adding NaI and acetone, thereby creating a third mixture, fluxing said third mixture, cooling it to room temperature, and extracting a product from said fluxed third mixture by extraction between (1) dichloromethane, diethyl ether, or ethyl acetate, and (2) water, (e) adding said product from step (d) to a reaction vessel, with (1) t-butyl carbazate, (2) water soluble carbonate or bicarbonate, and (3) a polar aprotic organic solvent, thereby creating a fourth mixture, stirring and refluxing said fourth mixture above room temperature for 10-24 hrs, and extracting between (1) dichloromethane, diethyl ether, or ethyl acetate dichloromethane and (2) H₂O to obtain an organic layer, drying said organic layer with MgSO₄ or Na₂SO₄, and removing solvent to obtain a crude solid mixture (“fifth mixture”), (f) adding an acid selected from trifluoracetic acid (“TFA”), glacial acetic acid, glacial formic acid, trichloroacetic acid to said fifth mixture, and then adding an acetoacetic hydrocarbon ester, thereby creating a sixth mixture, adding said sixth mixture to a solution of a weak base, thereby creating a seventh mixture, which seventh mixture has a pH, adjusting said pH of said seventh mixture to 3-7 with any combination of sodium acetate, Na₂CO₃, NaHCO₃, NH₄Cl, NaOH, HCl or TFA, thereby creating an eighth mixture, extracting said eighth mixture with dichloromethane to obtain an organic layer, and drying said organic layer over MgSO₄ or Na₂SO₄, thereby obtaining a crude product of 7-EMCMP, thereby synthesizing 7-EMCMP. In some embodiments, the weak base of step (f) is Na₂CO₃. In some embodiments, the methods further comprise step (g), triturating said crude product of 7-EMCMP with ethyl acetate at or near its boiling temperature, followed by dropwise addition of diethyl ether to precipitate 7-EMCMP, thereby obtaining purified 7-EMCMP.

In yet another group of embodiments, the invention provides a compound, 1-[7-(-4-methylcoumarin)]-3-methyl-5-pyrazolone (“7-MCMP”), said compound having the structure:

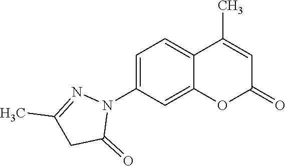 [SMILES: CC1=NN(c2ccc3c(C)cc(=O)oc3c2)C(=O)C1]

In still a further group of embodiments, the invention provides methods for releasing, labeling, and, optionally, analyzing, O-glycans present on a selected glycoconjugate of interest with 7-MCMP, said method comprising: incubating in a container said selected glycoconjugate of interest in an aqueous solution comprising 7-MCMP, at a temperature of 0° C. to 100° C. for a time between about 10 minutes and 23 hours. In some embodiments, the aqueous solution further comprises an effective amount of a base. In some embodiments, the base is dimethylamine, triethylamine (“TEA”), sodium hydroxide, potassium hydroxide, lithium hydroxide, and ammonium hydroxide. In some embodiments, the base is TEA. In some embodiments, the TEA is present at a concentration of 0.5M to 5M. In some embodiments, the 7-MCMP is present at a concentration of 0.05M to 5M. In some embodiments, the glycoconjugate is incubated in said aqueous solution at a temperature of 0-100° C. for a time between about 10 minutes and 23 hours. In some embodiments, the selected glycoconjugate of interest is present in said solution in an amount between 4 μg and 200 μg. In some embodiments, the selected glycoconjugate of interest is a glycoprotein or a glycopeptide. In some embodiments, the aqueous solution contains no hydrazine. In some embodiments, the temperature at which said aqueous solution is incubated is 70-90° C. In some embodiments, the container is a multi-well plate. In some embodiments, the multi-well plate is a 96-well plate.

In yet another group of embodiments, the invention provides kits for releasing and labeling O-glycans present on a glycoconjugate of interest, said kits comprising: (a) one or more containers, (b) a base selected from dimethylamine, triethylamine (“TEA”), sodium hydroxide, potassium hydroxide, lithium hydroxide, and ammonium hydroxide, or a combination of two or more of these, and (c) a fluorescent O-glycan labeling compound, said compound comprising a pyrazolone core, a linker, and, a coumarin core, wherein (1) said linker covalently links said pyrazolone core to said coumarin core, and (2) when said pyrazolone core is reacted with an O-glycan to label said O-glycan, thereby forming an O-glycan labeled with a derivative of said O-glycan labeling compound, said O-glycan labeled with said derivative of said O-glycan labeling compound is detectable by fluorescence. In some embodiments, the fluorescent O-glycan labeling compound is a compound of:
Structure 3,

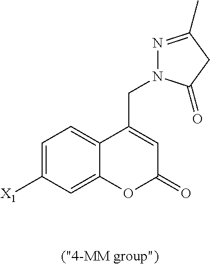

wherein X₁=Cl, Br, I, OH, OMe, OEt, NH₂, NMe₂, NEt₂, of
   Structure 4

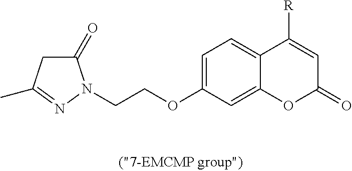

wherein R=CH₃ or CF₃, of
Structure 5,

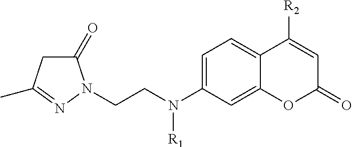

wherein R₁=H or CₙH₂ₙ₊₁; R₂=CH₃ or CF₃, of
Structure 6,

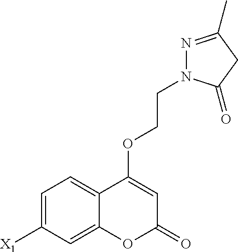

wherein X₁=H, Cl, Br, I, OH, OMe, OEt, NH₂, NMe₂, NEt₂, of
Structure 7

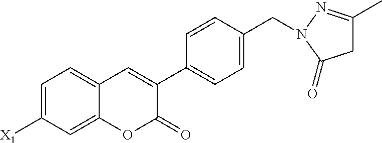

wherein X₁=H, Cl, Br, I, OH, OMe, OEt, NH₂, NMe₂, NEt₂, or of
Structure 8,

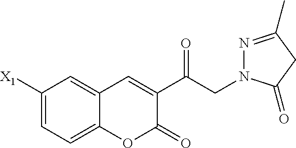

wherein X₁=H, Cl, Br or I. In some embodiments, the fluorescent O-glycan labeling compound is a compound of Structure 3, and has the structure of Structure 1:

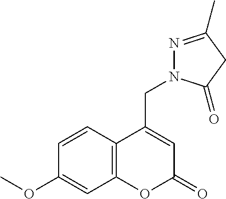 [SMILES: COc1ccc2c(CN3N=C(C)CC3=O)cc(=O)oc2c1]

(“4-MM”). In some embodiments, the fluorescent O-glycan labeling compound is a compound of Structure 4, and has the structure:

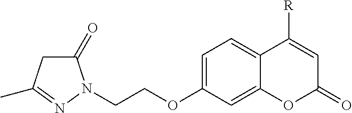

wherein R=CH₃ (“7-EMCMP”). In some embodiments, the base is TEA. In some embodiments, the base and said label are in separate compartments of the same container. In some embodiments, the kits further comprise: (d) at least one O-glycan standard.

In still another group of embodiments, the invention provides kits for releasing and labeling O-glycans present on a glycoconjugate of interest, said kit comprising: (a) one or more containers, (b) a base selected from dimethylamine, triethylamine (“TEA”), sodium hydroxide, potassium hydroxide, lithium hydroxide, and ammonium hydroxide, or a combination of two or more of these, disposed in one of said containers, and (c) a label, 1-[7-(-4-methylcoumarin)]-3-methyl-5-pyrazolone (“7-MCMP”), disposed in one of said containers. In some embodiments, the base is TEA.

### BRIEF DESCRIPTION OF DRAWINGS

FIG. 1 presents the structures of the compounds referred to herein as “7-MCMP” and as “4-MMOCMP” (or, more conviently, as “4-MM”). The Figure shows the numbering of the atoms of the respective compounds and provides guidance on functional aspects of the structures.

FIG. 2 is a graph presenting an extended ion chromatogram comparing the MS signal of the standard O-glycan label referred to as “PMP” to that of an exemplar 0-glycan label of the invention, referred to as “4-MMOCMP.” The structures of the respective labels (referred to in the legend at the top left as “dye”; the two terms are used interchangeably for purposes of this disclosure) are shown about the trace representing the signal from that label.

FIG. 3 is a graph showing the extended ion chromatograms for an exemplar O-glycan, 3′-sialyllactose (“3′-SL”) labeled with PMP and for the same O-glycan labeled with an exemplar O-glycan label of the invention, 4-MMOCMP, under the same conditions. As can be seen from the Figure, the signal from the 3′-SL labeled with 4-MMOCMP is lower than the signal from the 3′-SL labeled with PMP, but the two signals are in the same order of magnitude.

FIG. 4 is a graph showing the extended ion chromatograms of an exemplar O-glycan, 3′SL, labeled with either (1) PMP or (2) 4-MMOCMP, in either (a) wholly aqueous solution or (b) 50% aqueous solution and 50% methanol. As can be seen, the presence of the methanol did not significantly affect the labeling of the exemplar O-glycan compared to labeling it with either label in a wholly aqueous solution.

FIG. 5 is a graph showing the detection by UV (top line, labeled “Absorbance Trace”), by fluorescence (middle line, labeled “Fluorescence Trace”) and by MS (bottom line, labeled “Extracted Ion Chromatogram”), of an exemplar O-glycan, 3′SL, labeled with 4-MMOCMP. (The offset between the trace for the MS signal compared to those for the UV signal and for the fluorescence signal is due to the physical characteristics of the instrumentation used to introduce the sample into the mass spectrometer.)

FIG. 6 is a graph showing results from labeling 40 micrograms of maltodextrin with 0.5 M 4-MM in the presence of 2.9 M TEA. The top line shows the detection of fluorescence signals for labeled D-glucose polymers of different degrees of polymerization (“DPs”). The next three lines (each labeled “Extracted Ion Chromatogram” and identifying the particular DP whose Extracted Ion Chromatogram is presented in that line) show the corresponding MS signals for the three labeled DPs presented in the Figure. The offset between the trace for the MS signal for each DP compared to that for the fluorescence signal from each DP is due to the physical characteristics of the instrumentation used to introduce the sample into the mass spectrometer. Seven DPs were labeled and detected in the study; to assist the reader in seeing the results in the graph, only the results for the first three DPs are shown in the Figure.

FIG. 7 presents two graphs showing the MS signals from a study in which O-glycans from bovine fetuin were released and labeled by an exemplar label of the invention, 4-MM. The top graph shows the Extracted Ion Chromatogram from a sample containing 400 micrograms of bovine fetuin, while the bottom graph shows the Extracted Ion Chromatogram from a sample containing 40 micrograms of bovine fetuin, an order of magnitude less fetuin than in the study whose sample is analyzed in the top graph. The glycans were labeled under the same conditions in both studies, with only the amount of bovine fetuin being changed. A comparison of the Extracted Ion Chromatograms presented in the two graphs shows that the same O-glycans were detected in the sample containing only 40 micrograms of bovine fetuin as were detected in the sample containing 400 micrograms.

### DETAILED DESCRIPTION

### Introduction

As stated in the Background section, the particular post translational modifications on a protein or peptide can significantly affect characteristics critical to its function. Accordingly, it is important in many therapeutic and industrial uses to determine which post translational modifications are present on a protein or peptide whose biological activity is of interest.

One exemplar post translational modification is the addition of one or more carbohydrate moieties, or glycans, to the protein or peptide. Current protocols for determining the glycans present on a glycoprotein, glycopeptide, peptidoglycan, or proteoglycan of interest typically involve releasing the glycans from the glycoconjugate, labeling the released glycans, and then analyzing the labeled glycans to determine which are present and in what relative or absolute quantities. (Glycoproteins, glycopeptides, peptidoglycans, or proteoglycans are sometimes collectively referred to herein as “glycoconjugates.” In preferred forms, the glycoconjugate is a glycoprotein or a glycopeptide). Glycans attached to a glycoconjugate through an N-glycosidic bond (“N-glycans”) can generally be released by enzymatic digestion under mild conditions. The release of glycans attached to the glycoconjugate by an O-glycosidic bond (“O-glycans”) is more problematic. While some simple glycans can be released by enzymatic digestion, releasing more complex O-glycans typically requires the use of strong bases or, more commonly, anhydrous hydrazine, a highly toxic and unstable chemical, under relatively harsh conditions.

N-glycans released from glycoconjugate by enzymatic digestion are typically released as glycosylamines, which can be labeled with labels, such as InstantPC® (Agilent Technologies, Inc., Santa Clara, CA), that are detectable by both fluorescence and by mass spectrometry (“MS”). Such labels allow the N-glycans released from the glycoconjugate to be quantitated by detecting the fluorescent signal of the labeled glycans (or, more precisely, of the derivatives of the glycans created by reacting the glycans with the label), and the molecular species of the N-glycans (or, more precisely, of the derivatives of the glycans created by reacting the glycans with the label) by MS analysis. Unfortunately, the label commonly used for labeling O-glycans, 1-phenyl-3-methyl-5-pyrazolone, or “PMP,” does not provide the capability to detect O-glycans labeled with it by both fluorescence and MS. While PMP does form derivatives of O-glycans with a signal that makes the derivative detectable by MS, PMP does not also make the derivative of the O-glycan detectable by fluorescence. No reagent is currently known in the art for labeling O-glycans released from glycoproteins or glycopeptides that makes the resulting labeled derivatives detectable by fluorescence and MS. Preferably, a label with such properties would also make the labeled derivatives detectable by ultraviolet light (“UV”).

The present disclosure reports the surprising discovery of compounds that label O-glycans released from glycoproteins, glycopeptides, peptidoglycans, or proteoglycans of interest and that creates derivatives that can be detected by fluorescence, by UV, and by MS. While, like PMP, the inventive labels can be used in “one-pot” procedures, unlike PMP, they permit quantitating the O-glycans released from the glycoconjugate by fluorescence as well as detecting the molecular species present by MS. Further, the inventive compounds can be used in multi-well plates and in high-throughput procedures. Thus, the inventive compounds provide a combination of properties that make them surprisingly superior to the labels currently available in the art for labeling O-glycans.

For clarity, it is noted that, once an O-glycan in a sample has been labeled by a label, whether an already-known label such as PMP or one of the inventive labels provided in this disclosure, it is formally no longer an O-glycan but rather a derivative of an O-glycan, attached to a derivative of whatever compound was used to label the O-glycan. As the purpose of the labeling procedure, however, is to detect O-glycans present in the sample, determine which ones they are and, preferably, to quantitate the O-glycans, practitioners routinely refer to the compound resulting from labeling the O-glycan with the label being used as a labeled O-glycan. This disclosure follows this art-recognized usage. Further, as this disclosure is directed to the labeling of O-glycans, rather than of N-glycans, references below to labeling glycans by the inventive compounds are understood to refer to the labeling of O-glycans, unless otherwise stated.

### Overview and Structure of the Inventive Labels

As noted, 1-phenyl-3-methyl-5-pyrazolone, or “PMP,” is currently perhaps the label most commonly used to label O-glycans. Unfortunately, while labeling O-glycans with PMP renders them detectable by MS, it does not render them detectable by fluorescence. This disclosure reports the results of efforts to find reagents that can be used to label O-glycans that result in labeled O-glycans that can be detected by MS, UV, and, preferably, by fluorescence, and, in preferred forms, that can be substituted for PMP in current workflows without requiring extensive changes in the workflow to which practitioners are currently accustomed, including the rapid and convenient “one-pot” procedures disclosed in co-owned International Patent Application PCT/US2020/064337, published as International Publication No. WO2021/119333 (the “'337 PCT application”), the contents of which are hereby incorporated by reference. In brief, like the procedures taught in the '337 PCT application, the release and labeling can be performed on small amounts of the glycoprotein, glycopeptide, peptidoglycan, or proteoglycan of interest, in small volumes of reagents, in a single step in a single container, do not require the use of inert gases, do not require a drying down step or a solid phase extraction step to concentrate the labeled glycans before providing them to an analytic means, do not require a separate labeling step, and can be performed without the use of hydrazine or the strong bases that have been used in many of the previous protocols for releasing and labeling O-glycans.

The discovery of substituted pyrazolones that have fluorescence was not straightforward. A number of chemistries were tried. It was found that some labels that were fluorescent by themselves were not fluorescent when conjugated to pyrazolone to create a compound that would form a covalent bond with an O-glycan. Other labels were fluorescent after conjugation to pyrazolone, but were not fluorescent when the pyrazolone-label conjugate was then bonded to an O-glycan. Accordingly, one could not predict from any particular molecule that simply because it exhibited fluorescence when not attached to a pyrazolone that it would continue to exhibit fluorescence when attached to a pyrazolone. Further, one could not predict that, simply because a pyrazolone-label conjugate was fluorescent, it would continue to exhibit fluorescence when it was in turn conjugated to an O-glycan. Surprisingly, studies undertaken in the course of developing the present disclosure resulted in the discovery of a class of structurally and functionally related compounds that fill this need in the art.

7-MCMP was the first pyrazolone found to retain fluorescence both when it was first conjugated to a pyrazolone and then when the label-conjugated pyrazolone (7-MCMP) was conjugated to a carbohydrate with a reducing end, such as an O-glycan released from a glycoconjugate. Even 7-MCMP, however, was found to have limitations as a label: it provides fluorescence to O-glycans if they are labeled with one molecule of 7-MCMP (labeling an O-glycan molecule with a single molecule of a label is sometimes referred to herein as “single-labeling”), but not if they are labeled with two molecules of 7-MCMP (labeling an O-glycan molecule with two molecules of a label is sometimes referred to herein as “double-labeling”). Labeling of an O-glycan with two molecules of a pyrazolone-coumarin label tends to be the result if the O-glycan is labeled at the temperatures used in standard workflows, while single-labeling can be the result of conducting the labeling at a temperature lower than the standard temperatures.

Studies undertaken with labeling O-glycans with 7-MCMP suggested both the possible cause of the difference in fluorescence seen between single-labeled and double-labeled O-glycans, and the solution. This discovery led to the development of pyrazolone-coumarin conjugates that remain fluorescent both when they single-label an O-glycan and when they double-label an O-glycan. The labels of this group allow O-glycans labeled with these labels to be detected by MS, by UV, and by fluorescence, regardless of whether the O-glycans are single-labeled or double-labeled.

The inventive labels can be described by Formula 1, below:

P-Lₙ-C, Formula 1,

wherein “P” designates a pyrazolone derivative, “L” denotes a linker in which “n” equals 0 or 1, and “C” represents a coumarin derivative. Each of these components of Formula 1 will be described further below.

Turning first to the pyrazolone derivative, the pyrazolone derivatives used in the inventive labels are derivatives (or “substituted”) forms of 3-methyl-5-pyrazolone, in which the pyrazolone is substituted at the nitrogen at position 1 by either a coumarin derivative, as in the exemplar compound referred to below as “7-MCMP” (an embodiment in which the “n” in Formula 1 equals 0), or by a linker atom or molecule (in embodiments in which the “n” in Formula 1 equals 1), which covalently bonds the pyrazolone derivative through the linker to the coumarin derivative.

For clarity, it is noted that, as the pyrazolone moiety of Formula 1 has been covalently attached to either the linker or to the coumarin derivative as described above, it is no longer formally 3-methyl-5-pyrazolone, but rather a substituted or derivatized form thereof. For convenience of reference, however, the substituted 3-methyl-5-pyrazolone of the labels disclosed herein will generally be referred to as “pyrazolone” or “the pyrazolone core,” except in the portions of the specification specifically discussing 3-methyl-5-pyrazolone as a starting material for synthesizing one of the inventive labels or otherwise specified.

Turning to the component “L” in Formula 1, in some embodiments, the “n” of the Lₙ term of Formula 1 equals zero. In these embodiments, no linker is present and the pyrazolone core is covalently attached directly to a carbon atom in the coumarin moiety, which is designated in Formula 1 by the letter “C.” 7-MCMP is an exemplar of labels in which n=0. Labels in this group are expected to provide fluorescence to O-glycans singly-labeled with the label.

In other embodiments, the “n” of the Lₐ term of Formula 1 equals 1, and a linker is present L designates a linker which can be (a) an alkyl chain of up to ten carbon atoms, or (b) a chain of up to ten members, which members can be carbon atoms or bridging groups, provided (i) that there is at least one carbon atom between the pyrazolone core and any bridging in the chain, and (ii) provided that at least one unsubstituted carbon atom is disposed between any two bridging groups in the chain. By way of example, the linker can be a chain of eight carbon atoms and two bridging groups, for a total of ten members in the chain, a chain of nine carbon atoms and one bridging group, for a total of ten members in the chain, a chain of two carbon atoms and one bridging group, for a total of three for a total of three members of the chain, a chain of two carbon atoms, for a total of two members in the chain, or a single carbon atom. In preferred embodiments, the bridging groups are independently selected from an ester group, an amine group, an alkyl group, a benzyl group, and a thioester group. Structures 1 and 3 through 8, below, show exemplary linkers that can be used to make different embodiments of labels within the scope of this disclosure. A section is provided below to provide more detailed teachings on bridging groups that can be used in constructing some embodiments of the inventive labels.

Turning to the coumarin portion of Formula 1, Wikipedia states:
“Coumarin . . . or 2H-chromen-2-one is an aromatic organic chemical compound with formula C₉H₆O₂. Its molecule can be described as a benzene molecule with two adjacent hydrogen atoms replaced by a lactone-like chain —(CH)═(CH)—(C═O)—O—, forming a second six-membered heterocycle that shares two carbons with the benzene ring. It can be placed in the benzopyrone chemical class and considered as a lactone.”

For convenience of reference, the non-heterocyclic ring of the coumarin compounds and coumarin derivatives discussed herein is generally referred to herein as the phenyl ring, while the heterocyclic ring is referred to as the lactone ring. A number of coumarin compounds are known, many of which are commercially available. The compounds generally differ by the substituents (such as methyl groups or carbonyl groups) present on the different carbons in the two rings, other than the two carbons shared by the two rings.

As with pyrazolone, once a coumarin molecule has been covalently bonded either directly to the pyrazolone core, or to a linker covalently bonded to the pyrazolone core, it is no longer formally considered a coumarin molecule, but rather to be a substituted coumarin or a derivative of coumarin (the terms “substituted coumarin” and “derivative of coumarin” are generally used interchangeably herein). For convenience of reference, however, the substituted coumarin portions of the labels disclosed herein will generally be referred to as “coumarin” or “the coumarin core.”

It is believed that any of the commercially available coumarin compounds can be derivatized to make a fluorescent O-glycan label of the present invention, so long as the starting coumarin compound is fluorescent. Any particular coumarin compound can be readily tested for its ability to serve as the coumarin core of a fluorescent label for O-glycans by following the teachings of the exemplar syntheses set forth in the Examples for synthesizing a pyrazolone-coumarin label (with the coumarin core either directly bonded to the pyrazolone core or conjugated to it by a linker as described above), reacting the resulting compound with an exemplar O-glycan, such as mannose, and testing to see if the labeled O-glycan is fluorescent. If it is, it can be used as a fluorescent label for O-glycans.

Finally, as noted above, 7-MCMP, the first label developed in the course of the work reported herein, is fluorescent when an O-glycan is singly labeled with it. As the singly-labeled glycan remains in the solution, however, it tends to be labeled by a second 7-MCMP molecule, and the label does not remain fluorescent when two labels are attached to the glycan. Thus, practitioners wishing to have the labeled glycan retain fluorescence when the glycan is double-labeled should manage the labeling reaction to reduce or eliminate double-labeling.

7-MCMP is a label in which the pyrazolone core is directly bound to the coumarin core. Tests using an exemplar label having a spacer between the pyrazolone core and the coumarin core showed that the exemplar label (sometimes referred to herein as “4-MM”) showed that the exemplar label remained fluorescent when the glycan was double-labeled by the label. 4-MM is an exemplar of the embodiments described by Formula 1 when the “n” of the “Lₙ” term of Formula 1 is 1. As double-labeling of the target O-glycans can be performed with less adjustment to currently used standard “one-pot” workflows and provides a stronger signal than does single labeling of the O-glycans, labels of Formula 1 in which the “n” equals 1 are preferred embodiments for labeling O-glycans in the inventive methods and kits for performing them.

4-Mm and Other Pyrazalone-Coumarin Molecules that Enable Detection of Double-Labeled O-Glycans by Fluorescence

As noted above, it was found that O-glycans that have been labeled with one molecule of 7-MCMP are fluorescent, but O-glycans labeled with two molecules of 7-MCMP are not. The basis for the absence of fluorescence observed in O-glycans that have been double-labeled with 7-MCMP was not known. Thus, it was not known how to make a label that would provide fluorescence to O-glycans that were double-labeled (thus allowing the labeled glycans to be made at the temperatures usually used for labeling reactions in standard workflows), while still making the labeled glycans detectable by MS and by UV.

Surprisingly, a solution to this problem has been discovered. A second-generation label was developed that allows O-glycans that are double-labeled by the label to be detected by fluorescence, MS, and UV, and that can be used in place of PMP in one-pot procedures for releasing and labeling O-glycans. Further surprisingly, the solution provides a genus of structurally and functionally related pyrazolone core-linker-coumarin core compounds that can be used to provide fluorescence to O-glycans labeled with them. Finally, in the course of the studies working with these new reagents for labeling O-glycans, methods of synthesis were found for producing the exemplar new pyrazolone-conjugated coumarin cores in greater quantities than was possible with the original methods. Each of these surprising discoveries will be discussed in turn.

### A. 4-MM

In one aspect, the present disclosure relates to the discovery of a first compound which allows practitioners to label O-glycans in “one-pot” procedures and which results in a labeled product that can be detected by fluorescence, MS, and UV. This first compound is comprised of a pyrazolone core covalently bound to a carbon which in turn is covalently bound to a carbon in the lactone ring of a coumarin core, thereby forming the compound 1-(4-methyl-7-methoxycoumarin)-3-methyl-5-pyrazolone, usually referred to herein as “4-MMOCMP” or as “4-MM.” The structure of 4-MMOCMP is set forth in Structure 1, below, along with some of its physical properties.

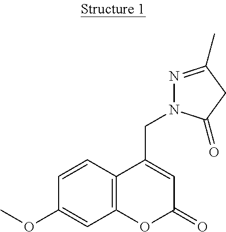 [SMILES: COc1ccc2c(CN3N=C(C)CC3=O)cc(=O)oc2c1]

### 1-(4-methyl-7-methoxycoumarin)-3-methyl-5-pyrazolone (“4-MMOCMP” or “4-MM”)

Chemical Formula: C₁₅H₁₄N₂O₄
   Exact mass: 286.0954
      MW: 286.2870

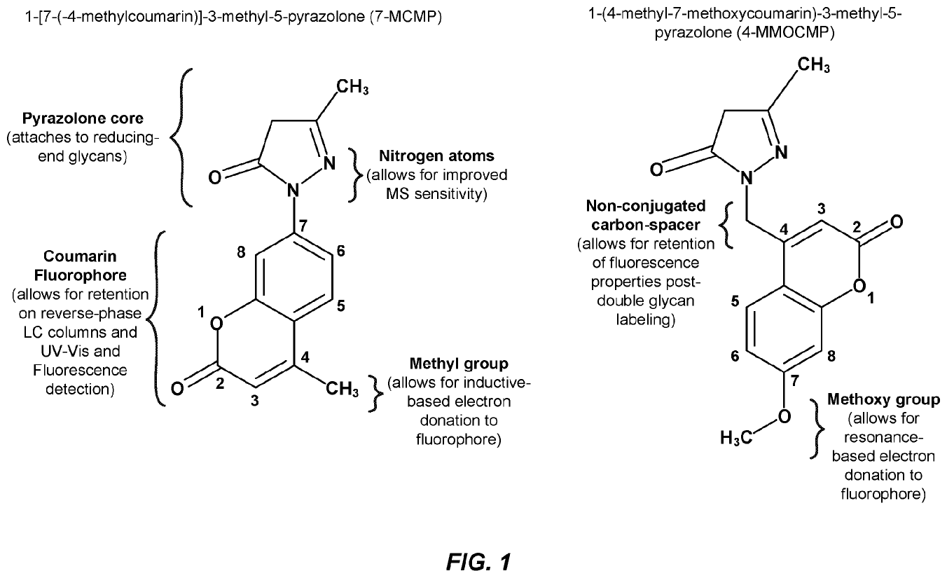

Studies using a physiologically important O-glycan, mannose, as an exemplar glycan, demonstrated that the label not only permitted detection of mannose labeled with 4-MM by MS, but that the label also retained fluorescence when two 4-MM molecules were bonded to a single O-glycan molecule (as noted above, an O-glycan molecule labeled by two molecules of a label is sometimes referred to herein as having been “double labeled”). A presumed structure for 4-MM is set forth as FIG. 1.

O-glycans that have been double labeled by 4-MM can be detected both by MS and by fluorescence, as well as by UV. Double labeling of O-glycans allows the practitioner to detect of O-glycans that may be present in the sample in only small quantities. And, because both the free dye and O-glycans labeled with the dye are fluorescent, it is easy to detect both the free dye and the labeled O-glycans when a sample from a one-pot procedure is subjected to separation by column chromatography and the analytes coming off the column are analyzed for fluorescence.

The structure of the originally developed label, 7-MCMP, is compared to the structure of 4-MM in FIG. 1. The structure of 4-MM was designed to differ from that of 7-MCMP by the introduction of a non-conjugated carbon spacer between the pyrazolone moiety and the coumarin core. Without wishing to be bound by theory, it is believed that electron flow around the rings is responsible for the label's fluorescence. Again, without wishing to be bound by theory, it is believed that introducing at least one non-conjugated carbon between the pyrazolone and the coumarin core creates a spatial separation that avoids interfering with the electron flow around the rings and thereby allows the label to retain fluorescence. And, further without wishing to be bound by theory, it is believed that, in the absence of the “carbon spacer,” electron flow is disrupted when a glycan is bonded to the pyrazolone moieties of two labels at the same time, as appears to be the case when the pyrazolone moieties of two 7-MCMP molecules bind the same glycan.

The starting material used to provide a carbon spacer to make 4-MM compared to the starting material used to make 7-MCMP resulted in some changes to where the pyrazolone core is attached to the coumarin core and in the substituent on the coumarin core of 4-MM compared to that of 7-MCMP. Without wishing to be bound by theory, it is believed that the difference in fluorescence of O-glycans double-labeled by 4-MM compared to 7-MCMP is not due either to the fact that, in 7-MCMP, the pyrazolone core is attached to the phenyl ring in the coumarin core, while in 4-MM, it is attached through the linker to the lactone ring, or that 7-MCMP has a carbon in the lactone ring is substituted with a methyl while in 4-MM, a carbon in the phenyl ring is substituted with a methyl group, as neither change would be expected to affect the electron flow around the rings.

Conveniently, 4-MM can be substituted for PMP in previously known protocols for releasing O-glycans from a glycoprotein, glycopeptide, peptidoglycan, or proteoglycan of interest and then labeling them, including the rapid and convenient “one-pot” procedures disclosed in co-owned International Patent Application PCT/US2020/064337, published as International Publication No. WO2021/119333 (the “'337 PCT application”), the contents of which are hereby incorporated by reference.

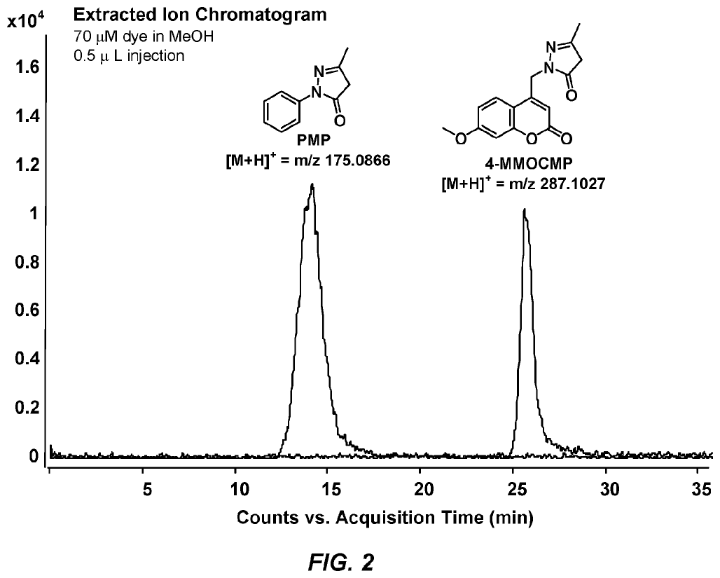

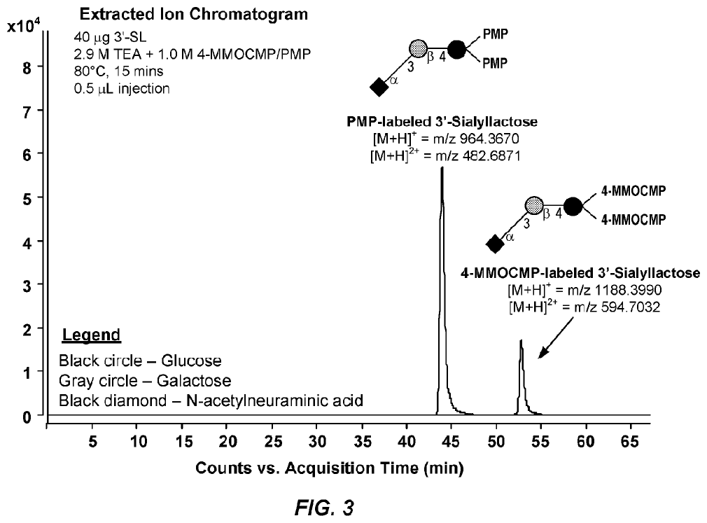

A study was conducted to compare the MS signal of 4-MM to that of PMP. As shown in FIG. 2, the MS signal of the two labels is similar. A further study was conducted to compare the MS signal of an exemplar O-glycan, 3′-sialyllactose (“3′-SL”) after it was labeled with 4-MM to that of the same amount of 3′-SL after it had been labeled with PMP under the same conditions. As shown in FIG. 3, the MS signal of 3′-SL labeled with 4-MM was lower than that of the same glycan labeled with PMP, but, importantly, the signal was of the same order of magnitude, indicating that O-glycans that can be detected by MS when they are labeled with PMP will also be detected when they are labeled with 4-MM. A study of the pKₐ of the two labels indicated that the pKₐ of PMP is ˜7.86, while that of 4-MM is ˜7.61, or a difference of ˜0.25. It is believed that the modest difference in labeling efficiency between the two labels is because 4-MM is slightly more acidic than PMP.

As in the procedures taught in the '337 PCT application, the release and labeling of O-glycans using 4-MM can be performed on small amounts of the glycoprotein, glycopeptide, peptidoglycan, or proteoglycan of interest, in small volumes of reagents, in a single step in a single container, do not require the use of inert gases, do not require a drying down step or a solid phase extraction step to concentrate the labeled glycans before providing them to an analytic means, do not require a separate labeling step, and can be performed without the use of hydrazine or the strong bases that have been used in many of the previous protocols for releasing and labeling O-glycans. In some preferred embodiments, the release and labeling reactions are performed in wholly aqueous solutions.

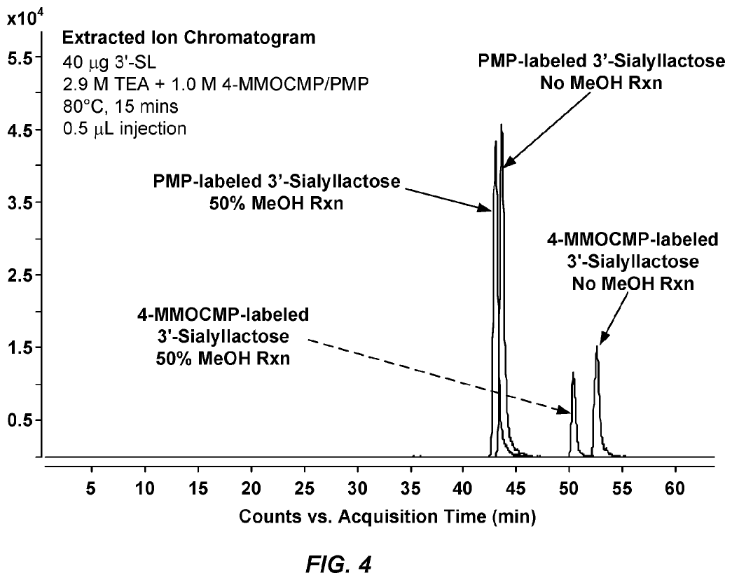

The results of a study in which an exemplar O-glycan, 3′-SL, was labeled with PMP or with 4-MM, in either water or in a 50% solution of water and an exemplar polar aprotic solvent, methanol, are shown in FIG. 4. As can be seen in FIG. 4, the presence of the organic solvent did not significantly affect the labeling of the O-glycan with either label when the organic solvent constituted 50% of the solution.

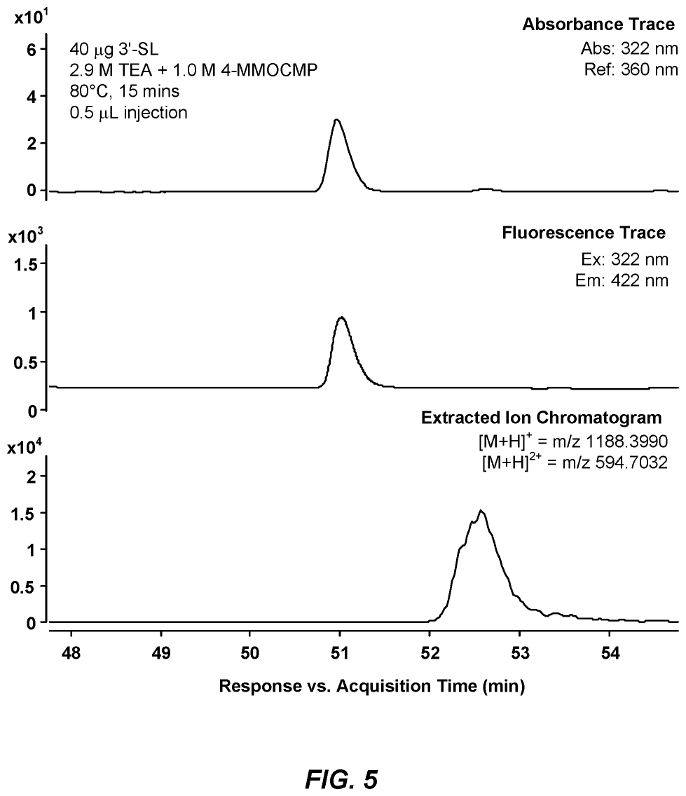

A study was conducted to determine whether an exemplar O-glycan, 3′-SL, labeled with 4-MM could be detected by UV, fluorescence, and MS. The results are shown in FIG. 5. As shown in FIG. 5, the labeled O-glycan could be detected by UV (the trace labeled “Absorbance Trace”), by fluorescence (the trace labeled “Fluorescence Trace”), and by MS (the trace labeled “Extracted Ion Chromatogram”). It is noted that the MS trace in FIG. 5 is offset from the other two traces because of the physical characteristics of the instrumentation used to introduce the sample into the mass spectrometer.

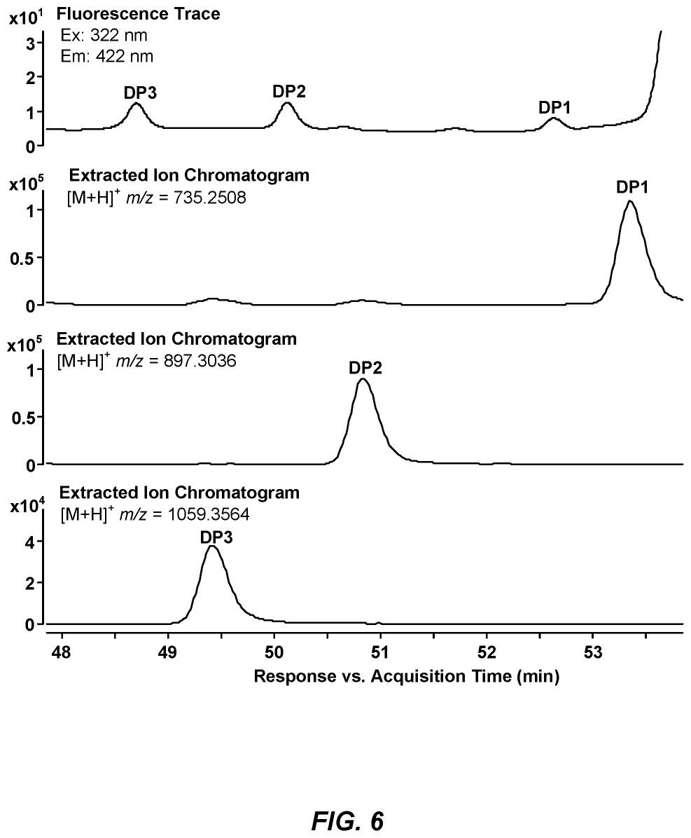

Further, a study conducted using maltodextrin, which is made of a mixture of chains of D-glucose units of variable length, showed that the “ladder” of polymerized glucose units of maltodextrin labeled with 4-MM could be detected both by fluorescence and by MS. FIG. 6 shows the fluorescence trace and MS chromatogram for the first three degrees of polymerization (“DPs”) of maltodextrin of seven DPs detected by the study (the other four were omitted from FIG. 6 for the purpose of presentation). As noted for FIG. 5, the MS traces of the DPs in FIG. 6 are offset from the fluorescence trace of the same DPs because of the physical characteristics of the instrumentation used to introduce the sample into the mass spectrometer.

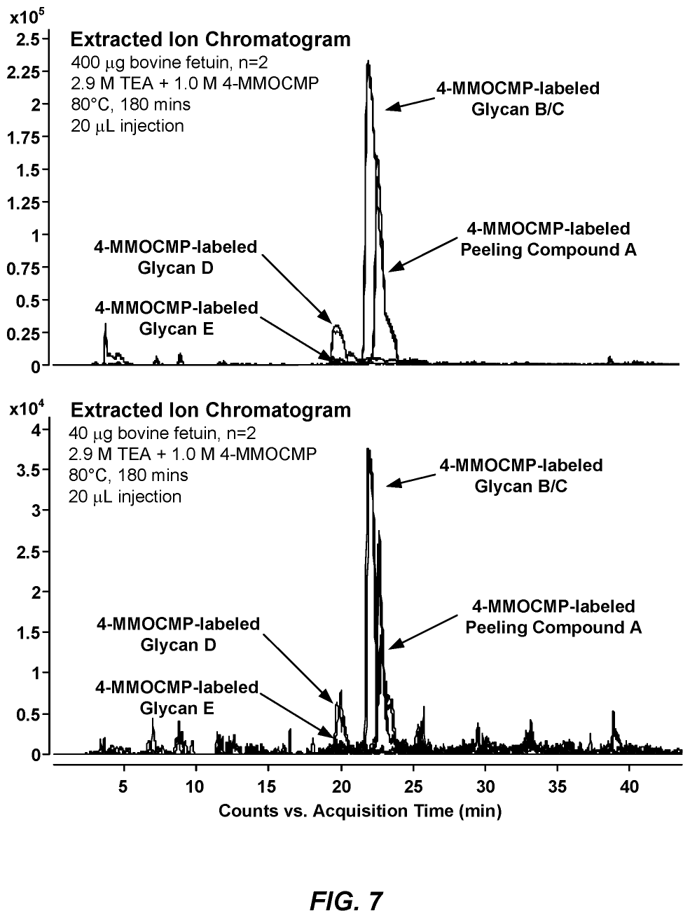

A study was conducted testing the ability of 4-MM to label glycans known to be present in bovine fetuin, using samples in which the amount of bovine fetuin present differed by an order of magnitude (400 micrograms and 40 micrograms). The results are shown in FIG. 7. As set forth in FIG. 7, the glycans in the fetuin were released, labeled, and detected by MS at both concentrations of 4-MM. Other studies with 4-MM confirmed that it could be used to label and detect exemplar O-glycans using samples with 40 micrograms of glycan or glycans.

The ability of 4-MM and, it is expected, the other labels provided in this disclosure, to be detected by fluorescence, MS, and UV, is expected to be make 4-MM and the other labels provided by this disclosure surprisingly better as labels for labeling O-glycans than those currently available in the art.

### B. First Method for Synthesizing 4-MM

To synthesize 4-MM, the following reaction scheme was developed:

### Reaction Step 1

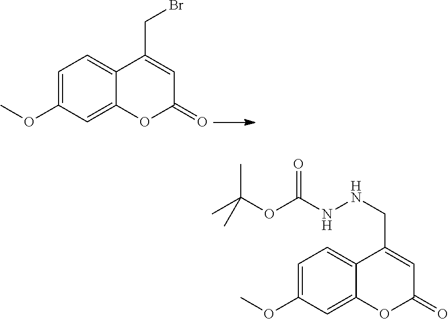 [SMILES: COc1ccc2c(CBr)cc(=O)oc2c1 ; COc1ccc2c(CNNC(=O)OC(C)(C)C)cc(=O)oc2c1]

To a round-bottom flask was added 4-Bromomethyl-7-methoxycoumarin (1.35 g, 5 mmol), t-butyl carbazate (1.32 g, 10 mmol), sodium carbonate (1.06 g, 10 mmol) and tetrahydrofuran (“THF,” 15 mL). The mixture was stirred and refluxed at 75° C. for 6 h. The mixture was gravity filtered. The solution was rotavapped to remove the solvent.

### Reaction Step 2

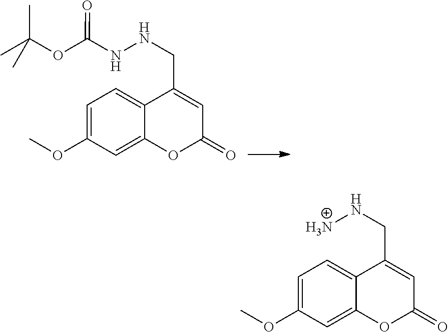 [SMILES: COc1ccc2c(CNNC(=O)OC(C)(C)C)cc(=O)oc2c1 ; COc1ccc2c(CN[NH3+])cc(=O)oc2c1]

To the round-bottom flask containing the crude solid mixture from the previous step (without further purification), methanol (15 mL) was added, and the mixture was heated to 50° C. to dissolve the solids. To the stirring solution, 12M HCl (7.5 mL) was added, and the solution was stirred at 50° C. for another 30 minutes. The mixture was vacuum filtered to collect the solids.

### Reaction Step 3

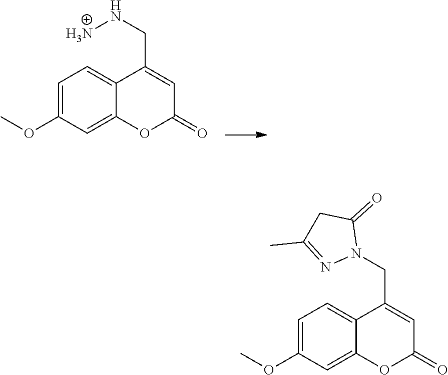 [SMILES: COc1ccc2c(CN[NH3+])cc(=O)oc2c1 ; COc1ccc2c(CN3N=C(C)CC3=O)cc(=O)oc2c1]

To a round bottom flask was added the filter cake of solids from the previous step (without further purification), ethanol (15 mL), ethyl acetoacetate (1.91 mL, 15 mmol) was added, and the mixture was heated to 80° C. under stirring for 16 h. The mixture was then concentrated with vacuum, diluted with DI water (15 mL) and extracted with ethyl acetate (15 mL) three times. The organic layer was dried over Na₂SO₄ and concentrated. The oily mixture was then purified by preparative liquid chromatography. Overall yield was 180 mg (13%). (This method of synthesizing 4-MM is sometimes referred to herein as the “original method of synthesizing 4-MM.”)

### C. Synthesis That More Than Doubles The Yield of 4-MM

The yield of 4-MM provided by the original method of synthesizing 4-MM developed to synthesize it was 13%. It would be desirable to have a method of synthesizing the compound that provided a higher yield to reduce costs and the quantity of reagents needed to make a given amount of 4-MM. Surprisingly, a more efficient method of synthesizing 4-MM was found that increases the yield of product by almost 240%. (This second, more efficient method of synthesizing 4-MM is sometimes referred to herein as the “improved method of synthesizing 4-MM” or “the second method of synthesizing 4-MM”).

### Reaction Step 1

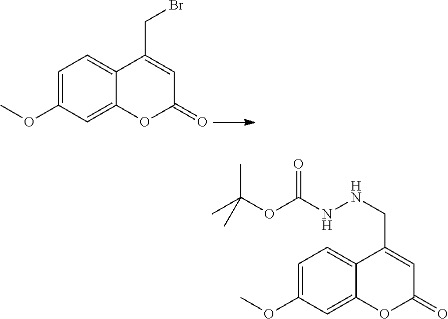 [SMILES: COc1ccc2c(CBr)cc(=O)oc2c1 ; COc1ccc2c(CNNC(=O)OC(C)(C)C)cc(=O)oc2c1]

4-Bromomethyl-7-methoxycoumarin (2.69 g, 10 mmol) was added to a round-bottom flask containing t-butyl carbazate (1.58 g, 12 mmol), sodium bicarbonate (1.26 g, 15 mmol), and tetrahydrofuran (10 mL). The mixture was stirred and refluxed at 65° C. for 16 h (monitored by thin layer chromatography (“TLC”), using Ethyl Acetate:Hexane=1:1, reactant Rf˜0.70, major product Rf˜0.45). The mixture was gravity filtered. The solution was optionally rotavapped to remove at least a portion of solvent, and then extracted between dichloromethane (40 mL) and H₂O (20 mL×2). The organic layer was dried over MgSO₄ or Na₂SO₄, gravity filtered. The solvent was then removed by rotavap.

This step is preferably conducted at a temperature of about 40° C. or more, and in a polar aprotic solvent. If using a solvent with a higher boiling point, such as dimethyl or dimethyl sulfoxide, the reaction temperature is preferably not higher than about 90° C., to reduce side reactions. “About” in the context of temperatures in this paragraph means±2° C. Excess t-butyl carbazate can optionally be removed from the crude reaction mixture by extraction using dichloromethane, diethyl ether or ethyl acetate, against saturated sodium bicarbonate.

### Reaction Step 2

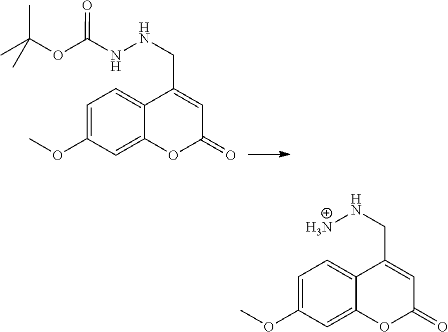 [SMILES: COc1ccc2c(CNNC(=O)OC(C)(C)C)cc(=O)oc2c1 ; COc1ccc2c(CN[NH3+])cc(=O)oc2c1]

To the round-bottom flask containing the crude solid mixture from the previous step (without further purification) was added trifluoroacetic acid (“TFA,” 10 mL, 131 mmol). The solution was stirred for 1 h at r.t. (monitored by TLC, Ethyl Acetate:Hexane=1:1, reactant Rf˜0.45, major product Rf˜0). The reaction mixture was used directly in the next step (“one-pot”).

### Reaction Step 3

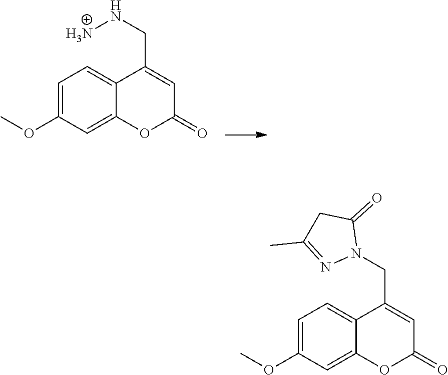 [SMILES: COc1ccc2c(CN[NH3+])cc(=O)oc2c1 ; COc1ccc2c(CN3N=C(C)CC3=O)cc(=O)oc2c1]

To the mixture from the previous step, ethyl acetoacetate (1.54 mL, 12 mmol) was added. The mixture was stirred for 3-16 h at r.t. (monitored by TLC, Ethyl Acetate, reactant Rf˜0, desired product Rf˜0.5, major side product Rf˜0.9). The mixture was added into a solution of Na₂CO₃ (5.30 g, neutralizing 100 mmol H⁺) in H₂O (40 mL). The solution was pH adjusted to 3-7 with any combination among sodium acetate, Na₂CO₃, NaHCO₃, NH₄Cl, NaOH, HCl or trifluoroacetic acid (“TFA”). The mixture is extracted with dichloromethane (30 mL×4). The organic layer was dried over MgSO₄ or Na₂SO₄, gravity filtered. The solvent was then removed by rotavap.

The crude product is yellow to orange in color, as a combination of solid and very viscous liquid. The crude product is triturated with ethyl acetate (4.5 mL) at or near its boiling temperature, followed by dropwise addition of diethyl ether (5 mL) to precipitate the majority of the desired product. The mixture was cooled to r.t., and solid collected by vacuum filtration, with rinsing with ethyl acetate:diethyl ether=2:3. The product was TLC pure, while ˜5% impurities may be found by LC-FLD. Overall yield was 892 mg (31%).

It is expected that the same synthetic route can be used to form other 4-MM variant pyrazolone-coumarin labels.

### D. Labels Other Than 4-MM In Which A Linker is Used to Attach Pyrazolone to a Coumarin Core

In another aspect, the invention provides other labels comprising a pyrazolone which is linked to a coumarin core through by a linker comprising more than one atom.

As previously noted, 4-MM has the structure of Structure 1:

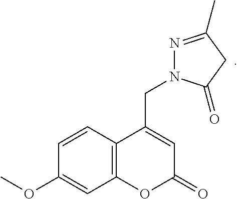 [SMILES: COc1ccc2c(CN3N=C(C)CC3=O)cc(=O)oc2c1]

As discussed further below, 7-MCMP has the structure of Structure 2:

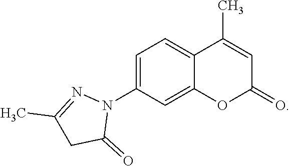 [SMILES: CC1=NN(c2ccc3c(C)cc(=O)oc3c2)C(=O)C1]

A comparison of the two molecules reveals that, in 7-MCMP, the pyrazolone is linked to the coumarin core at a carbon in the phenyl ring distal to the lactone ring, while in 4-MM, the pyrazolone is linked to the coumarin core at a carbon in the lactone ring that, in 7-MCMP, bears a methyl group (and that no methyl group is present in 4-MM). Further, 4-MM has an ether group on the phenyl ring at the position to which the pyrazolone is attached in 7-MCMP.

In one aspect, the invention relates to compounds having a structure which is the same as that of 4-MM, which at the position designated by the term “X₁” in Structure 3, below, one of a group of selected electronegative substituents. (The letter “X” is used in this structure and in other structures below in its usual use in organic chemistry to denote an electronegative group). The compounds of Structure 3 are sometimes referred to herein as the “4-MM genus.”

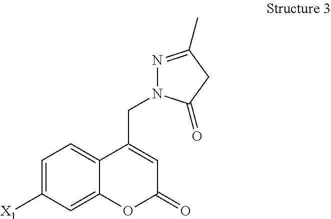

“4-MM group”
X₁=Cl, Br, I, OH, OMe, OEt, NH₂, NMe₂, NEt₂

The members of the 4-MM group can be synthesized following the synthesis for 4-MM, using a starting coumarin compound that will provide the desired substituent at the position designated as “X₁.”

In another aspect, the invention provides other labels that can be synthesized from a commercially available coumarin compound other than the one used as the starting material for the synthesis of 4-MM. This is convenient not only because it allows making labels other than 4-MM without having to synthesize a starting material from scratch, but also because it allows the practitioner to select from among various coumarin compounds depending on factors such as cost, ease of handling and storage, and availability from the practitioner's preferred vendors. The selected coumarin compounds can then be linked to a pyrazolone following the teachings herein to create a label that allows detecting glycans labeled with the label by MS, fluorescence, and UV.

In one aspect, the invention relates to the development of a label that is sometimes referred to herein as “7-EMCMP” (not to be confused with 7-MCMP), in which the pyrazolone is linked to the coumarin core through two carbons and an ether group. This label has the structure of Structure 4, below, where the R group is CH₃.

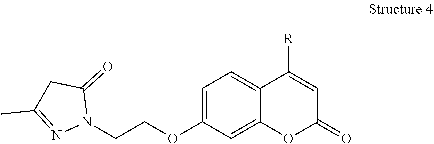

“7-EMCMP group”
R=CH₃ or CF₃.

A method of synthesizing 7-EMCMP is set forth in the Examples. The compounds of Structure 4 are sometimes referred to herein as the “7-EMCMP group.” It is contemplated that the person of skill can readily make the trifluoro species of the 7-EMCMP group by following the synthetic pathway set forth below for 7-EMCMP.

In another aspect, the invention relates to labels having the structure set forth in Structure 5, below.

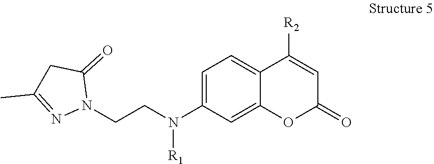

R₁=H or CₙH₂ₙ₊₁; R₂=CH₃ or CF₃.

The synthesis of members of this group can be made following the synthetic pathway set forth in the Examples for synthesizing 7-EMCMP.

In another aspect, the invention relates to labels having the structure set forth in Structure 6, below.

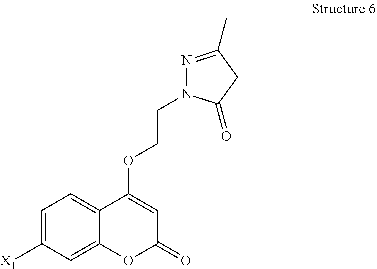

X₁=H, Cl, Br, I, OH, OMe, OEt, NH₂, NMe₂, NEt₂.

As with the group above, the synthesis of members of this group can be made following the synthetic pathway set forth in the Examples for synthesizing 7-EMCMP.

In another aspect, the invention relates to labels having the structure set forth in Structure 7, below.

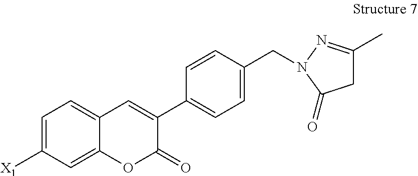

X₁=H, Cl, Br, I, OH, OMe, OEt, NH₂, NMe₂, NEt₂.

Members of this group can be made following the synthetic pathway set forth herein for synthesizing 4-MM.

In another aspect, the invention relates to labels having the structure set forth in Structure 8, below.

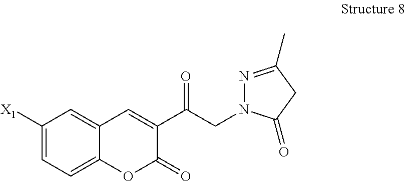

X₁=H, Cl, Br or I.

Members of this group can be made following the synthetic pathway set forth herein for synthesizing 4-MM. The carbonyl group in the linker linking the pyrazolone and the coumarin core could over time be attacked by the pyrazolone, reducing the effectiveness of the label at labeling O-glycans. Thus, it is preferred that labels having the structure of structure 8 be used shortly after synthesis, such as within one month, two weeks, or one week.

Finally, in another aspect, the invention provides labels in which the pyrazolone is linked to any of the coumarin cores shown above through a linker of two carbons, three carbons, or four carbons. As practitioners will appreciate, as the chain of carbons in the spacer becomes longer, the molecule will become less hydrophilic and will gradually become less useful in labeling glycans in an aqueous solution. Thus, in some preferred embodiments, the pyrazolones is connected to the coumarin core through a linker of three or fewer carbons.

### 7-MCMP and its Properties

The first of the inventive labels developed in the course of the work described herein is 1-[7-(-4-methylcoumarin)]-3-methyl-5-pyrazolone (sometimes referred to herein as “7-MCMP”). In addition to providing 7-MCMP as a label, the present disclosure provides methods of making it. Studies undertaken to synthesize 7-MCMP in quantities allowing better studies of its properties in labeling O-glycans resulted in the development of a synthetic pathway that dramatically increased the yield of the final product compared to the method originally developed to synthesize 7-MCMP. Accordingly, the present disclosure provides 7-MCMP itself, methods of making 7-MCMP, methods of using 7-MCMP to label O-glycans, and kits comprising 7-MCMP.

The ability to detect and analyze by UV and MS O-glycans labeled with 7-MCMP, and by fluorescence, for O-glycans singly-labeled by 7-MCMP, is expected to make 7-MCMP a label useful in labeling and analyzing O-glycans. Conveniently, with one change in the procedure, discussed in more detail below, 7-MCMP can be substituted for PMP in previously known protocols for releasing O-glycans from a glycoprotein, glycopeptide, peptidoglycan, or proteoglycan of interest and then labeling them, including the rapid and convenient “one-pot” procedures disclosed in co-owned International Patent Application PCT/US2020/064337, published as International Publication No. WO2021/119333 (the “'337 PCT application”), the contents of which are hereby incorporated by reference. In brief, like the procedures taught in the '337 PCT application, the release and labeling can be performed on small amounts of the glycoprotein, glycopeptide, peptidoglycan, or proteoglycan of interest, in small volumes of reagents, in a single step in a single container, do not require the use of inert gases, do not require a drying down step or a solid phase extraction step to concentrate the labeled glycans before providing them to an analytic means, do not require a separate labeling step, and can be performed without the use of hydrazine or the strong bases that have been used in many of the previous protocols for releasing and labeling O-glycans.

As noted, the inventive compound is 1-[7-(-4-methylcoumarin)]-3-methyl-5-pyrazolone (sometimes referred to herein as “7-MCMP”). 7-MCMP has the structure shown in Structure 2:

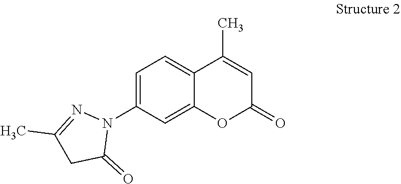 [SMILES: CC1=NN(c2ccc3c(C)cc(=O)oc3c2)C(=O)C1]

As can be seen from Structure 2, 7-MCMP has a substituted pyrazolone (the 5-membered heterocyclic ring on the left with two adjacent nitrogen atoms) bonded to a coumarin molecule which has been substituted with a methyl group on the heterocyclic ring. 7-MCMP is a compound of Formula 1 in which the “n” of the Lₙ term equals zero.

As noted earlier, the double ring structure of coumarin after it has been attached to the pyrazolone moiety will sometimes referred to herein as a “coumarin core,” with the non-heterocyclic ring sometimes referred herein to as the “phenyl ring” and the heterocyclic ring of the coumarin sometimes referred to herein as the “lactone ring.”

When detected by MS, 7-MCMP has a m/z of 257.0921. When detected by fluorescence, 7-MCMP has a maximum excitation wavelength of 335 nm, and a maximum emission wavelength of 417 nm. (For clarity, it is noted that the mass-to-charge ratio, or “m/z” of glycans labeled with 7-MCMP will be different from that of 7-MCMP itself, since the mass of a glycan labeled with 7-MCMP will of course be larger than that of the starting 7-MCMP molecule. The excitation and emission wavelengths of the 7-MCMP label, however, will be the same.)

Like PMP, 7-MCMP can be reacted with glycoproteins, glycopeptides, peptidoglycans, or proteoglycans of interest in the presence of one or more bases, such as triethylamine, and heated to release and label O-glycans. Like PMP, released O-glycans labeled with 7-MCMP can be detected both by MS and by UV. Thus, in instances in which the practitioner wishes to be able to detect the presence of the labeled O-glycans by UV, MS, or both, 7-MCMP can simply be substituted for PMP in protocols for labeling released O-glycans with PMP. Labeling with 7-MCMP also adds the ability to detect by fluorescence the released O-glycans which have been labeled with 7-MCMP.

When labeling with PMP, each O-glycan molecule is preferably labeled with two PMP molecules. In studies underlying the present disclosure, however, it was found that O-glycan molecules labeled with two 7-MCMP molecules (“double-labeling”) were detectable by MS and by UV, but not by fluorescence. O-glycans labeled with a single 7-MCMP molecule could also be detected by fluorescence, in addition to UV and MS. Thus, O-glycans double-labeled with 7-MCMP can be detected by MS analysis and by UV, as is currently done with O-glycans labeled with PMP. In embodiments in which the practitioner wishes to detect O-glycans only by MS, by UV, or by both, as is currently done with PMP, 7-MCMP can be substituted for PMP in protocols for labeling O-glycans with PMP to provide 0-glycans that have been double-labeled.

To take advantage of the ability to detect a labeled glycan by MS, by UV, and by fluorescence, however, the reaction conditions for labeling O-glycans by PMP are adjusted to reduce or, preferably, eliminate, double labeling of the glycan with 7-MCMP. Since the reaction is thermodynamically controlled, this can be conveniently accomplished by reducing the temperature used for the release and labeling of the O-glycans. The release and labeling of O-glycans with PMP is typically performed at 80° C. Conducting the labeling reaction at 80° C. was found to result in almost all of the glycans being double-labeled, while conducting the labeling reaction at 50° C. resulted in some of the O-glycans being singly-labeled, and conducing the labeling reaction at 20° C. resulted in many of the O-glycans being singly-labeled.

Accordingly, when the practitioner wishes to obtain O-glycans that can be detected by fluorescence, UV, and MS, it is preferred that the O-glycans are labeled with 7-MCMP at temperatures between 0° C. and 40° C. If the practitioner wants to obtain O-glycans that can be detected by UV, and MS, and is not concerned with detecting them by fluorescence, the practitioner can, in addition to using a temperature within the range just stated (which will result in at least some of the O-glycans being singly labeled and that can be detected by fluorescence), but preferably labels the O-glycans at a temperature above 40° C., and preferably labels them at 80° C., which is the temperature at which O-glycans are labeled in protocols for labeling them by PMP. Labeling them at this temperature results in the O-glycans being double-labeled by 7-MCMP, which is expected to provide a stronger signal for detection by MS and by UV, but which does not allow them to be detected by fluorescence. Double labeling can also be performed at temperatures above 80° C., such as 85, 90, 95, or 100° C. At temperatures of 90° C. or above, the container holding the sample is preferably tightly covered to reduce or prevent evaporation of the water in the solution, which would change the concentration of the base and of the 7-MCMP label and would result in incomplete product formation.

Some of the studies underlying the present disclosure used bovine fetuin, a blood protein, as the source of O-glycans to be released, labeled, and analyzed. In other studies, mannose was used as a sample carbohydrate for labeling, as it has a reducing end that can be labeled by 7-MCMP.

As noted in an earlier section, an O-glycan that has been labeled with a label, such as 7-MCMP, is no longer chemically an “O-glycan” but is rather a chemical entity derived from the O-glycan which now bears a portion of the label, such as 7-MCMP, which allows the new chemical entity to be detected by, for example, MS and UV. It is further noted that, technically, the term “glycan” refers to a carbohydrate covalently attached to a glycoconjugate, such as a glycoprotein. It bears repeating, however, that the purpose of labeling O-glycans released from a glycoconjugate of interest, such as a glycoprotein, glycopeptide, peptidoglycan, or proteoglycan, however, is to determine which O-glycans were present on the glycoconjugate and, preferably, to determine the amount of each O-glycan present on the glycoconjugate. As a convenient shorthand, therefore, practitioners continue to refer to O-glycans that have been released from a glycoprotein as O-glycans, and continue to refer to released O-glycans that have been labeled, such as with PMP or with 7-MCMP, as “labeled O-glycans,” as a labeled form of the particular O-glycan that was released from the glycoconjugate and subjected to labeling, or just as the glycan whose presence in the original sample was determined by analyzing the labeled glycan. In accordance with this practice in the art, O-glycans that have been released from a glycoconjugate and derivatized with a label, such as 7-MCMP, are sometimes referred to in this disclosure as “labeled O-glycans,” as “O-glycans labeled with” or “by” one of the labels discussed herein, or by similar terms.

Similarly, once a label, such as 7-MCMP, has reacted with an O-glycan, it is no longer chemically the starting chemical entity but rather some chemical derivative thereof. Nonetheless, for convenience of reference, compounds resulting from reacting 7-MCMP with a glycan will sometimes be referred to herein as a glycan having been “labeled with 7-MCMP” and compounds resulting from reacting a glycan with a label discussed herein will sometimes be referred to herein as the glycan having been labeled with that label.

In sum, 7-MCMP and the inventive methods and kits using it provide the ability to analyze O-glycans present on a glycoprotein, glycopeptide, peptidoglycan, or proteoglycan of interest by MS and UV, but also adds the ability to detect O-glycans singly-labeled with 7-MCMP by fluorescence.

For convenience of reference, the phrases “selected glycoconjugate” or “glycoconjugate of interest” are sometimes used herein to refer to a member of the group consisting of glycoproteins, glycopeptides, peptidoglycans, and proteoglycans. For convenience of reference, reference herein to “glycoproteins” also includes “glycopeptides” unless otherwise specified or required by context. Finally, it is noted that “glycoconjugate of interest,” “glycoprotein of interest,” and the like refer to any glycoconjugate or any particular glycoconjugate bearing O-glycans which the practitioner wishes to release and label. For example, the glycans present on production runs of glycoproteins approved for therapeutic use are typically analyzed to confirm the consistency of the glycosylation of the glycoproteins remains during the run. In such an instance, the therapeutic glycoprotein is the “glycoprotein of interest.” Other terms not specifically defined in this disclosure are intended to have the meaning as those terms are used in the art.

### 7-MCMP Synthesis and Use

In addition to its labeling properties, another advantage of 7-MCMP is that, since it is a pyrazolone, it can be used in place of 1-phenyl-3-methyl-5-pyrazolone (“PMP,” CAS number 89-25-8) in the high-throughput methods taught in the '337 PCT application referenced above. The parameters set forth in the '337 PCT application, either as is, or with modest adjustments, allow for the rapid, high-throughput release of O-glycans, and labeling the released O-glycans with great sensitivity. These methods allow the user to prepare labeled O-glycans in just 15 minutes in either single vial or 96-well plate format, and facilitate automated sample preparation. Some of the reaction conditions explained in the '337 PCT application useful in labeling O-glycans with 7-MCMP are set forth in the next section.

As noted, PMP was the label used in the studies reported in the '337 PCT application. Glycans are labeled by PMP by Michael addition under alkaline conditions to avoid the loss of sialic acids which could occur under acidic conditions. See, e.g., Ruhaak et al, Anal Bioanal Chem. 2010; 397(8): 3457-3481. Glycans labeled by PMP can be detected by MS, typically after the glycans have been separated by liquid chromatography. See, e.g., Lottova et al., J Amer. Soc. Mass. Spectrom., 2005; 16(5):683-96. This sequence of analytic procedures is sometimes referred to as “liquid chromatography/mass spectrometry,” or “LC/MS.”

The present disclosure regards labeling O-glycans with 7-MCMP, which, like PMP, allows the labeled glycans to be detected by UV and MS when the O-glycans are labeled with two molecules of 7-MCMP, but allows them to also be detected by fluorescence when the individual O-glycan molecules are labeled with one molecule of 7-MCMP.

The '337 PCT application discusses how to reconstitute PMP and to mix it with triethylamine (“TEA”) prior to use. Briefly, the '337 PCT application reported that adding a pre-mixed solution of TEA to dry PMP, and adding concentrated TEA to dry PMP, and then diluting the mixture with water gave equivalent results. The '337 PCT application also reported that powdered PMP could be weighed out to obtain the desired amount of PMP to be mixed, adding TEA as a liquid, and then adding a measured quantity of water calculated to result in a mixture with the desired molarity. The same procedures can be followed to obtain desired concentrations of 7-MCMP.

TEA is an organic compound that is not highly soluble in water; at molarities of 3.9 or higher, it will not mix completely, resulting in some separation of the aqueous and organic phases. Conveniently, starting stock solutions of TEA can be made which, when equal amounts of solution containing the glycoprotein sample are added, reduce the concentration by half (for example, a stock solution of 0.90M TEA would result in a solution of 0.45M TEA after the glycoprotein sample is added). For convenience of reference, the solution of TEA, 7-MCMP, and glycoprotein sample is sometimes referred to herein as the “release/labeling solution”.

The '337 PCT application reported that PMP made the solution more acidic as its concentration increased. As with PMP, therefore, the amount of 7-MCMP in the solution can be used to control the pH of the release/labeling solution. At more acidic pHs, lower amounts of O-glycans will be released from the glycoprotein for analysis, while at more basic pHs, the glycans are not labeled as well and can react further with the base and be destroyed. It is expected that, as was found for PMP, accurate glycan profiles can be obtained with final 7-MCMP concentrations ranging from 0.05M-5M.

Based on the results using PMP, it is believed that the molarity of 7-MCMP in the release/labeling solution is preferably between 0.05 and 5, between 0.10 and 4, between 0.20 and 3, between 0.35 and 2.5, between 0.50 and 2, between 0.75 and 1.75, between 0.85 and 1.5, with “about” in this context meaning 0.05M. In some embodiments, the molarity of 7-MCMP in the release/labeling solution is 1M.

The Examples below, sets forth two protocols for synthesizing 7-MCMP. The second protocol, set forth in Example 3, below, results in a surprisingly better yield of 7-MCMP than does the original protocol set forth in Examples 1 and 2

### Bridging Groups

Wikipedia explains electron donating functional groups in the context of an article on electron donating and electron withdrawing groups, which commences as follows:
“In electrophilic aromatic substitution reactions, existing substituent groups on the aromatic ring influence the overall reaction rate or have a directing effect on positional isomer of the products that are formed. An electron donating group (EDG) or electron releasing group (ERG, Z in structural formulas) is an atom or functional group that donates some of its electron density into a conjugated π system via resonance (mesomerism) or inductive effects (or induction)—called +M or +I effects, respectively—thus making the π system more nucleophilic. As a result of these electronic effects, an aromatic ring to which such a group is attached is more likely to participate in electrophilic substitution reaction. EDGs are therefore often known as activating groups, though steric effects can interfere with the reaction.
Electron donating groups are generally ortho/para directors for electrophilic aromatic substitutions, while electron withdrawing groups are generally meta directors with the exception of the halogens which are also ortho/para directors as they have lone pairs of electrons that are shared with the aromatic ring.”
   Wikipedia article entitled “Electrophilic aromatic directing groups,” accessed Feb. 26, 2023 (internal references omitted).

In some embodiments, the linkers of the inventive labels in which the “n” in the term Lₙ of Formula 1 is one can contain one or more of what are sometimes referred to herein as a bridging groups. The bridging groups are generally electron donating functional groups (called in the Wikipedia article an electron donating group).

In preferred embodiments, the bridging groups are selected from an ester group, an amine group, an alkyl group, a benzyl group, and a thioester group. The embodiments provided as the numbered Structures of this disclosure show some preferred linkers and preferred bridging groups for constructing labels within the scope of the disclosure. The suitability of any particular bridging group for use in constructing a label within the scope of the disclosure can be readily tested by synthesizing a label following the teachings of the exemplar synthetic techniques shown for 4-MM or 7-EMCMP, respectively, using the resulting candidate label to label an exemplar O-glycan, such as mannose, and determining if the O-glycan labeled by the candidate label is fluorescent. If it is, that particular bridging groups is suitable for making labels within the scope of this disclosure.

Bases for Releasing O-Glycans from Glycoconjugates

A variety of bases can be used to release O-glycans from a glycoconjugate, such as a glycoprotein. Suitable bases include dimethylamine, triethylamine (“TEA”), sodium hydroxide, potassium hydroxide, lithium hydroxide, and ammonium hydroxide. Concentrations of any particular base suitable for releasing O-glycans from any particular glycoconjugate of interest (sometimes referred to as an “effective concentration” of the base) can be readily determined by simply running parallel samples of the glycoconjugate of interest in an aqueous solution, with each sample containing a different concentration of the particular base or combination of bases, and measuring the amount of O-glycans released by standard methods, including those disclosed in the Background, and those disclosed in the '337 PCT application.

In preferred embodiments, the base is triethylamine, or “TEA,” CAS number 121-44-8. TEA is an organic compound commonly used as a base in organic syntheses. (The abbreviation “TEA” is also used in organic chemistry to refer to several other organic compounds; in this disclosure, however, “TEA” refers only to triethylamine unless otherwise stated.) TEA, along with other ammonium bases such as ammonium hydroxide (NH₄OH) and dimethylamine, or “DMA,” has been used before as a base in procedures to release and label O-glycans. The reports of the use of TEA in O-glycan release and labeling, prior to the '337 PCT application, however, reported using it at 0.2M in a 50% solution of hydrazine. See, Kisiel et al., Toxicology Mechanisms and Methods, 18:503-07 (2008); Kisiel, et al., Acta Biochemica Polonica, 1999, 46(3):753-757. Studies discussed in the '337 PCT application showed that hydrazine was not needed in releasing and labeling O-glycans from a glycoprotein, and that avoiding the use of that compound made the release and labeling process safer. Accordingly, hydrazine is preferably omitted in performing the methods discussed herein. If the practitioner wishes for some reason to include it, despite it not being necessary, it is preferred that the hydrazine not be present in a concentration of more than 5%.

Further, whereas the reports by Kisiel reported good results in releasing and labeling O-glycans after 48 hours of incubation, the methods disclosed in the '337 PCT application allowed the determination of the relative glycan profile of the glycoprotein in as little as 10 minutes and determination of the absolute glycan profile in as little as 15 hours. It is anticipated that single- and double-labeling of O-glycans with 7-MCMP will occur in time periods similar to those shown in the '337 PCT application for labeling with PMP.

Studies underlying the '337 PCT application demonstrated that TEA was a surprisingly better base for O-glycan release and labeling methods than either NH₄OH or dimethylamine (“DMA”). The use of TEA resulted in higher total glycan signal and comparable results regardless of whether water or acid was used to stop the release and labeling reactions. Studies underlying the present disclosure, however, indicated that TEA that had been exposed to air over a period of months lost some efficacy in the release and labeling procedure. Without wishing to be bound by theory, it is believed that TEA in containers that have been opened and partially used over a period of months absorbs some moisture from ambient air that enters the container as its contents are used over time, that oxygen reacts with the TEA, or both.

Accordingly, if TEA is used as the base in the release and labeling procedure, it is preferably from a container that was only opened within the last three months, the last two months, or the last month. In some embodiments, the TEA is from a container that is opened for the first time for use in the release and labeling procedure. In some embodiments, the present invention provides kits containing reagents for releasing and labeling O-glycans that include TEA as the base to assist in removing the glycans. It is anticipated that, in such kits, the TEA will be in a container sealed at the time of manufacture and accordingly will be fresh when opened for use in the release and labeling process.

### Glycoproteins, Glycopeptides, Peptidoglycans, and Proteoglycans

As discussed in the Introduction, the invention relates to compositions and methods for analyzing the O-glycans attached to a glycoprotein, glycopeptide, peptidoglycan, or proteoglycan (collectively, “glycoconjugates,” any particular one, a “glycoconjugate”) of interest. The glycoconjugate of interest can be in dry form, or can be suspended in an aqueous solution. One advantage of the methods taught in the '337 PCT application compared to most prior techniques is that only very small amounts of the glycoconjugate of interest is needed for analysis. Studies reported in the Examples of the '337 PCT application used glycoconjugate sample sizes as low as 10 μg. Typical sample sizes used in the studies were 40 μg. It is expected that 7-MCMP, 4-MM, or 4-MM variant pyrazolone-coumarin labels can be used for labeling sample sizes the same or similar to those disclosed in the '337 PCT application. As disclosed in that application for use with PMP, it is expected that, no matter the amount of glycoconjugate used as the sample (referring to sample sizes from 10 to 200 μg), 40 μL of water will be added to suspend the glycoconjugate and that this suspension will be then be added to 40 μL of the reaction mixture containing TEA and one of the labels disclosed herein to achieve the desired final concentration of TEA and of the particular label. Other amounts of water could, of course, be used to achieve a desired final concentration of TEA and of the label. In some embodiments, the glycoconjugate is a glycoprotein or a glycopeptide.

Based on the results of the studies in the '337 PCT application, it is believed that the amount of the selected glycoconjugate in the sample to have its O-glycans released and labeled with one of the labels disclosed herein is preferably between 4 μg and 200 μg, between 5 μg and 150 μg, between 5 μg and 125 μg, between 5 μg and 110 μg, between 5 μg and 105 μg, between 5 μg and 100 μg, between 5 μg and 90 μg, between 5 μg and 80 μg, between 5 μg and 70 μg, between 10 μg and 60 μg, between 10 μg and 50 μg, or about 40 μg, with “about” in this sentence meaning ±10 μg. In some embodiments, the amount of the selected glycoconjugate in the sample is about 40 μg, with “about” in this sentence meaning ±5 μg. In some embodiments, the amount of the selected glycoconjugate in the sample is about 40 μg, with “about” in this sentence meaning ±2 μg. In some embodiments, the amount of the selected glycoconjugate in the sample is 40 μg.

Methods using the labels disclosed herein are expected to work with glycoconjugates in which glycans are O-linked to amino acids. As noted above, such glycoconjugates include, in addition to glycopeptides and glycoproteins, peptidoglycans and proteoglycans. The inventive methods with respect to releasing O-glycans from a glycoconjugate are not expected to work with glycolipids and lipopolysaccharides, in which the O-glycan is not attached to an amino acid. Accordingly, as used herein, the term “glycoconjugate” comprises glycopeptides and glycoproteins, peptidoglycans and proteoglycans, but not glycolipids or lipopolysaccharides. In preferred embodiments, the glycoconjugates are glycopeptides and glycoproteins. As used herein, the terms “glycoconjugate of interest,” “glycoprotein of interest,” “glycopeptide of interest,” “peptidoglycan of interest,” and “proteoglycan of interest” refer to a compound of the designated type (e.g., a glycopeptide) as to which the practitioner wishes to analyze which O-glycans are conjugated to it.

### Time and Temerature Conditions for Labeling

As is well known in the art, a relative glycan profile gives the relative proportion of each glycan that was released from a selected glycoconjugate, while an absolute glycan profile quantitates the amount of each glycan released. One of the advantages of the methods disclosed in the '337 PCT application was the surprising speed with which a relative glycan profile of the O-glycans present on the selected glycoconjugate could be obtained. As reported in the Examples of the '337 PCT application, kinetic studies were performed to determine the minimal reaction time necessary to obtain accurate relative and absolute glycan profiles. Those studies indicated that, incubated at 80° C., an accurate relative glycan profile of the exemplar glycoconjugate could be obtained in as little as 10 minutes and was consistent at when samples were measured at timepoints up to 23 hours. Conversely, maximum glycan release was not seen until after 15 hours of incubation. Similar results are expected to obtain using the labels disclosed herein.

Thus, in uses in which the practitioner wishes to analyze the UV or MS signal of glycans labeled with one of the labels disclosed herein, the O-glycans can be released and incubated with the label at 80° C., to cause the O-glycans to be double-labeled with the label. As with PMP, in this situation, the incubation will provide an accurate relative glycan profile of the exemplar glycoconjugate in as little as 10 minutes, but can be incubated for up to 23 hours. For all of the labels disclosed herein other than 7-MCMP, the process just described will also allow the practitioner to analyze the fluorescence of the labeled O-glycans. For analyzing the fluorescence of O-glycans labeled with 7-MCMP, the practitioner will wish to have the O-glycans single-labeled with the 7-MCMP label. In this case, the practitioner should incubate the O-glycans with the TEA/7-MCMP release and labeling mixture at temperatures 10 degrees or more below 80° C.

### Methods of Synthesizing 7-MCMP

The inventive compound, 7-MCMP, was originally synthesized by the protocol set forth in Examples 1 and 2 (sometimes referred to herein as the “original method of 7-MCMP synthesis”). This synthesis of a novel compound is itself novel and inventive.

Reaction scheme 1, below, is a schematic setting forth the original inventive method for synthesizing 7-MCMP. The starting material for the inventive method is 7-amino-4-methylcoumarin (also known as “AMC”; CAS No. 26093-31-2), which is commercially available from a number of suppliers, including Sigma-Aldrich, Inc., St. Louis, MO (catalog no. 257370) and Cayman Chemical, Ann Arbor, MI (item no. 27792).

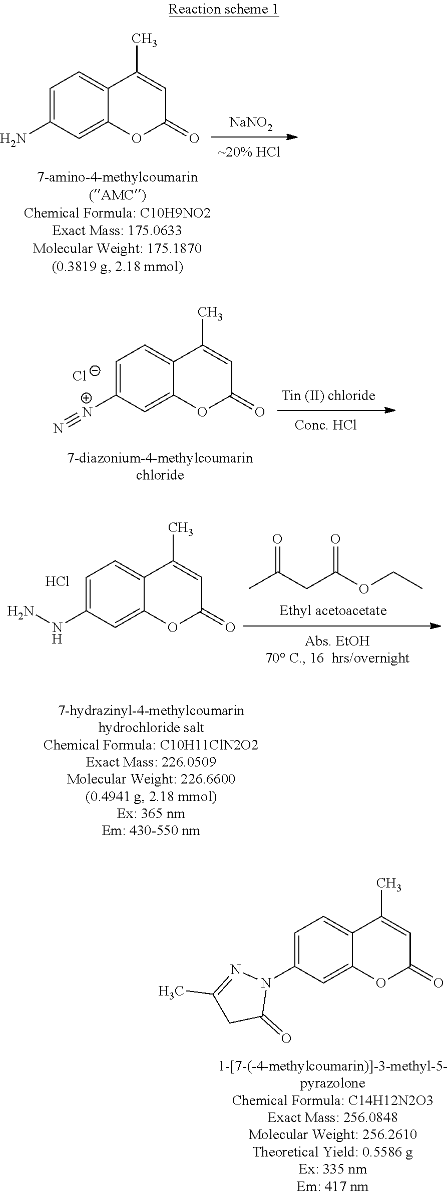

7-MCMP made by this method was used in the first studies exploring the properties of the compound. Surprisingly, however, a second method was developed that succeeded in producing 7-MCMP in a percentage yield that is dramatically larger—as much as 5 times the yield—than that of the original method of 7-MCMP synthesis. For ease of reference, the method that produces these notably higher quantities of 7-MCMP compared to the original method of synthesis is sometimes referred to herein as the “second 7-MCMP synthetic method.”

An exemplar set of reaction conditions and reagent amounts for synthesizing 7-MCMP by the second 7-MCMP synthetic method is set forth in detail in Example 3, below. The following provides a narrative description of the reaction scheme for the second 7-MCMP synthetic method.

As with the original 7-MCMP synthetic method, the starting material for the second 7-MCMP synthetic method is AMC. AMC is added to a solution of HCl in a reaction container set in an ice bath, with stirring of the solution. A chilled aqueous solution of sodium nitrate, NaNO₂, is added slowly to the AMC/HCl mixture while the temperature is maintained at ˜5° C. or lower, thereby forming a diazonium salt solution.

An aqueous solution of sodium carbonate is then slowly added to the diazonium salt solution until the mixture reaches pH 5-6. An aqueous solution of sodium sulfite (Na₂SO₃), preferably 22% sodium sulfite, is added and the resulting mixture is then removed from the ice bath, warmed to room temperature, stirred, and heated to 80° C. Concentrated HCl is then added to the mixture, and the mixture is heated and refluxed, during which time SO₂ is released. Following reflux, the mixture is cooled in an ice bath, resulting in the precipitation of 7-hydrazinyl-4-methylcoumarin hydrochloride salt, which can be collected by filtration. The salt is then placed in a reaction container, such as a flask, with ethanol, catalytic HCl, and ethyl acetoacetate, and the resulting mixture is then heated and allowed to reflux for 12 or more hours. (As known in the art, HCl is considered to be “catalytic” with respect to a reaction when it needs to be added in only small amounts to start the reaction and is then regenerated as the reaction progresses.) The solid product, 7-MCMP, is then isolated. Chemists are well familiar with methods for isolating solids from solutions, such as by filtration or by evaporation of the solvent, and it is contemplated that practitioners can readily use standard methods, such as by rotary evaporation, for isolating 7-MCMP, the solid produced by the reaction scheme described above.

As noted above, Examples 1-3 set forth in detail the original and the second 7-MCMP synthetic methods, including the amounts of the various reagents used. As persons of skill are aware, chemical syntheses proceed by reactions among atoms or molecules, with the reaction described as a balanced reaction setting forth. As persons of skill are aware, in many synthetic schemes, one or more of the reagents in a particular step are provided in excess the stoichiometric ratios of the respective reactants and products to permit the reaction of that step to proceed until one of the reagents, the limiting reagent, is completely consumed. In the reactions set forth in the two methods of synthesis, some of the reagents are provided in excess compared to the amount of AMC used as the starting material to yield as much of the desired product, 7-MCMP, as possible. As is common in chemistry, however, too large an excess of a reagent decreases the reaction rate and the product yield; thus, it is desirable to use a large enough excess of the other reagents to allow the desired reaction to proceed, but not so much as to reduce product yield below a point desired by the practitioner. Persons of skill are familiar with balancing these variables in any given reaction scheme and with selecting amounts of reagents to use based on their previous experience with similar reaction schemes. AMC is the starting material in both of the inventive methods of synthesizing 7-MCMP set forth herein. Once the practitioner has chosen the scale of the synthesis to be conducted by selecting the amount of AMC to be used as the starting material, it is expected that the practitioner can readily determine the amounts of the other reagents to be used in the respective synthetic methods by scaling them up or down proportionately to the amount of AMC to be used in the synthesis compared to the amount of AMC used in the studies reported in the Examples, below.

As noted in Reaction Scheme 1, sodium nitrite is reacted with AMC in a diazotization reaction that results in the formation of 7-diazonium-4-methylcoumarin chloride. Use of amounts of sodium nitrite lower than that of the AMC leads to an incomplete reaction, whereas amounts of sodium nitrite substantially higher than that of the AMC are unstable in the acidic conditions under which the reaction is conducted. Accordingly, the amount of sodium nitrite used to react with AMC to form 7-diazonium-4-methylcoumarin chloride is preferably equimolar to the amount of AMC used, or at a slight excess. In preferred embodiments, the amount of sodium nitrite is equimolar to the amount of AMC. In some embodiments, the amount of sodium nitrite to react with the AMC to form 7-diazonium-4-methylcoumarin chloride is, in moles, 1% to 10% more than the moles of AMC present in the reaction solution.

As noted in the description above, the diazotization reaction is conducted while the solution of the reagents is chilled. Preferably, the temperature of the solution is maintained at or below 10° C. but above negative 10° C. More preferably, the temperature is maintained within the range from and including 0° C. to and including 5° C.

In Example 3, step 6, sodium carbonate contributes twice the molarity of hydrogen ions (H⁺) as the molarity of the sodium carbonate that is added. (For example, 72 mmol Na₂CO₃ provides 144 mmol of H⁺). As noted in Example 3, step 6, sodium carbonate is added until the pH of the solution is at or between pH 5-6.

It is noted that the reaction contemplated when sodium sulfite is added to the diazonium salt solution in Example 3, step 8, requires 2 equivalents of sulfite, and an excess is preferred. As the excess sulfite is converted into SO₂ in step 9, the sodium sulfite is preferably provided at <5 equivalents (<2.5 fold excess). An excess of concentrated HCl is used to both release excess sodium sulfite as SO₂, and to ensure that all the hydrazine is precipitated as its hydrochloride salt form. As persons of skill are aware, concentrated HCl is typically sold as a solution with a concentration ranging from 36.5% to 38%, but for convenience is sometimes described herein as being 37% HCl. Use of concentrated HCl is preferred, as it reduces the volume of the reagent necessary, but lower concentrations (such as 20%-36.4%) can be used if the practitioner so desires. Finally, it is noted that some of the ethyl acetoacetate present during the conversion of the hydrochloride salt to the pyrazolone (as shown in Reaction Scheme 1) may be hydrolyzed. Thus, use of an excess of ethyl acetoacetate is preferred. Any excess of ethyl acetoacetate can be removed by, for example, evaporation.

### Kits

In some embodiments, the invention provides kits containing one or more of the labels disclosed herein for releasing, labeling, and, optionally, analyzing, O-glycans present on a glycoconjugate of interest. The kits typically comprise one or more containers, a base selected from dimethylamine, triethylamine (“TEA”), sodium hydroxide, potassium hydroxide, lithium hydroxide, ammonium hydroxide, or a combination of two or more of these, and one or more of the labels disclosed herein, such as 4-MM. In some embodiments, the base is TEA. In some embodiments, the base and the label or labels chosen to be included, such as 4-MM, are in separate compartments of the same container. In some embodiments, the base and the label or labels are in separate containers. In some embodiments, the kit further comprises at least one O-glycan the identity of which is known. The known O-glycan can be used as a standard in assays of O-glycans released from glycoconjugates of interest.

### EXAMPLES

### Example 1

This Example sets forth a protocol that can be used to synthesize a 7-hydrazinyl-4-methylcoumarin hydrochloride salt.

### Procedure

1) To a 100 mL pear-bottomed flask, add magnetic stir bar, 7-amino-4-methylcoumarin (0.3819 g, 2.18 mmol) and solvent (9 mL of water mixed with 16 mL of concentrated HCl) while stirring vigorously. The solution will appear milky-white in color.

2) After the addition of solvent, chill reaction mixture to 0° C. in an ice bath while stirring.

3) In a separate vessel, add 5.44 g NaNO₂ followed by 12 mL water. Once clear, add the sodium nitrite solution drop-wise to the reaction mixture via pipette. It is important to maintain the temperature of the reaction solution between 0-5° C. After adding a single drop of sodium nitrite solution, the reaction solution will instantly turn bright-yellow in color.

4) Remove addition funnel and stir reaction mixture at 0° C. for 1 hour.

5) Prepare 25 mL of 2 M stannous chloride/tin (ii) chloride solution in concentrated HCl (9.45 g SnCl₂ in 25 mL concentrated HCl). Cool the stannous chloride solution to 0° C. then add the solution drop-wise to the reaction mixture via pipette.

6) Add the stannous chloride solution drop-wise into the reaction mixture with vigorous stirring while maintaining the temperature of the reaction between 0-5° C. The moment the stannous chloride is added, precipitation of 7-hydrazinyl-4-methylcoumarin hydrochloride salt should occur. After the stannous chloride solution has been fully added, the reaction flask was capped and allowed to stand for 1 hour at 0° C.

7) After 1 hour, filter the precipitate and wash 3 times with concentrated hydrochloric acid. Collect filtered 7-hydrazinyl-4-methylcoumarin hydrochloride salt and dry in a vacuum desiccator.

### Example 2

This Example sets forth a protocol for synthesizing 7-MCMP from 7-hydrazinyl-4-methylcoumarin hydrochloride salt.

### Procedure

1) Transfer the collected/filtered 7-hydrazinyl-4-methylcoumarin hydrochloride salt (0.4941 g, 2.18 mmol) produced by the procedure of Example 1 to a 250 mL round-bottomed flask and add a magnetic stir bar, 40.95 mL of absolute ethanol, and 1.265 mL of ethyl acetoacetate and attach a water-condenser column to the reaction flask.

2) Heat reaction mixture to 70° C. in an oil-bath while stirring vigorously overnight (16 hours). Recover the dried material via rotary evaporation.

3) The theoretical yield of 1-[7-(-4-methylcoumarin)]-3-methyl-5-pyrazolone will be 0.5586 g.

### Example 3

Example 3 sets forth an inventive protocol that surprisingly increases the percentage yield of 7-MCMP by as much as 5 times compared to the percentage yield produced by the method taught in Examples 1 and 2.

1) In a 100 mL two-necked round-bottomed flask, add magnetic stir bar followed by 37% HCl (12 mL, 144 mmol H⁺). Place the reaction vessel to chill in an ice bath and set the stir bar to stir vigorously.

2) To this flask, add 7-amino-4-methylcoumarin (0.700 g, 4.0 mmol). A white suspension is formed.

3) Dissolve add sodium nitrite (NaNO₂, 0.333 g, 4.8 mmol) in 1.0 mL water. Chill the solution in an ice bath.

4) Under an isopropanol (10-15 wt %) ice bath (−5 to −10° C.), add the sodium nitrite solution from Step 3 dropwise into the 7-amino-4-methylcoumarin from Step 2. The solution temperature should be maintained ˜5° C. or lower. An alternative cooling mechanism may be used in scale-up, with −5° C. or lower cooling capability being recommended.

5) In a 50 mL centrifuge tube, prepare a slurry of sodium carbonate (Na₂CO₃, 7.63 g, 72 mmol) in 30 mL H₂O.

6) Add the Na₂CO₃ slurry/solution from step 5, dropwise to the diazonium salt solution from step 4. Stop addition when pH reaches 5-6.

7) Prepare a 22% sodium sulfite (Na₂SO₃, 2.02 g in 9.2 mL, 19.4 mmol) solution.

8) Add Na₂SO₃ from step 7 to the diazonium salt solution from Step 6 (fast addition is allowed). After the addition is completed, remove the ice bath and allow the solution to warm to room temperature and stir for 1 hour. Then heat to 80° C. for 15 min.

9) Add 37% HCl (2.5 mL) to the reaction mixture. The mixture bubbles and releases SO₂. The solution should now be strongly acidic. Heat and reflux for 30 min.

10) Cool the reaction mixture to ˜5° C. in an ice bath. Collect the precipitated white or lightly orange solids by filtration, it should be the 7-hydrazinyl-4-methylcoumarin hydrochloride salt.

11) To a round-bottomed flask add the 7-hydrazinyl-4-methylcoumarin hydrochloride salt, ethanol (40 mL), catalytic HCl, ethyl acetoacetate (2.55 mL, 2.6 g, 20 mmol). Heat to reflux overnight.

12) The mixture is orange colored solution with small amount white solids. Remove the solvent by rotary evaporation (“rotavap”). White solids forms in a dark orange liquid.

### Example 4

This Example sets forth a protocol for synthesizing 4-MM.

1. To a round-bottom flask was added 4-Bromomethyl-7-methoxycoumarin (1.35 g, 5 mmol), t-butyl carbazate (1.32 g, 10 mmol), sodium carbonate (1.06 g, 10 mmol) and tetrahydrofuran (“THF”) (15 mL). The mixture was stirred and refluxed at 75° C. for 6 hours. dThe mixture was gravity filtered. The solution was rotavapped to remove the solvent.

2. To the round-bottom flask containing the crude solid mixture from the previous step, methanol (15 mL) was added, and the mixture was heated to 50° C. to dissolve the solids. To the stirring solution, 12M HCl (7.5 mL) was added, and the solution was stirred at 50° C. for another 30 minutes. The mixture was vacuum filtered to collect the solids.

3. To a round bottom flask was added the filter cake of solids from the previous step (without further purification), ethanol (15 mL) and ethyl acetoacetate (1.91 mL, 15 mmol) was added, and the mixture was heated to 80° C. under stirring for 16 h. The mixture was then concentrated with vacuum, diluted with DI water (15 mL) and extracted with ethyl acetate (15 mL) for three times. The organic layer was dried over Na₂SO⁴ and concentrated. The oily mixture was then purified with preparative liquid chromatography. Overall yield was 180 mg (13%).

### Example 5

This Example sets forth a protocol for synthesizing 4-MM that surprisingly yields more than twice the percentage yield of 4-MM compared to the synthetic method set forth in Example 4.

1. To a round-bottom flask was added 4-Bromomethyl-7-methoxycoumarin (2.69 g, 10 mmol), t-butyl carbazate (1.58 g, 12 mmol), sodium bicarbonate (1.26 g, 15 mmol) and tetrahydrofuran (“THF”) (10 mL). The mixture was stirred and refluxed at 65° C. for 16 h. (monitored by TLC, Ethyl Acetate:Hexane=1:1, reactant Rf˜0.70, major product Rf˜0.45) The mixture was gravity filtered. The solution was optionally rotavapped to remove at least a portion of solvent then extracted between dichloromethane (40 mL) and H₂O (20 mL×2). The organic layer was dried over MgSO₄ or Na₂SO₄, gravity filtered. The solvent was then removed by rotavap.

2. To the round-bottom flask containing the crude solid mixture from the previous step (without further purification) was added trifluoroacetic acid (“TFA”, 10 mL, 131 mmol). The solution was stirred for 1 h at room temperature (“r.t.”), (monitored by thin layer chromatography (“TLC”, Ethyl Acetate:Hexane=1:1, reactant Rf˜0.45, major product Rf˜0). The reaction mixture was used directly in the next step (“one-pot”).

3. To the mixture from the previous step, ethyl acetoacetate (1.54 mL, 12 mmol) was added. The mixture was stirred for 3-16 h at room temperature (monitored by TLC, Ethyl Acetate, reactant Rf˜0, desired product Rf˜0.5, major side product Rf˜0.9). The mixture was added into a solution of Na₂CO₃ (5.30 g, neutralizing 100 mmol H+) in H₂O (40 mL). The solution was pH adjusted to 3-7 with any combination among sodium acetate, Na₂CO₃, NaHCO₃, NH₄Cl, NaOH, HCl or TFA. The mixture was extracted with dichloromethane (30 mL×4). The organic layer was dried over MgSO₄ or Na₂SO₄, gravity filtered. The solvent was then removed by rotavap.

4. The crude product is yellow to orange in color, as a combination of solid and very viscous liquid. The crude product was triturated with ethyl acetate (4.5 mL) at or near its boiling temperature, followed by dropwise addition of diethyl ether (5 mL) to precipitate the majority of the desired product. The mixture was cooled to r.t., and solid collected by vacuum filtration, with rinsing with ethyl acetate:diethyl ether=2:3. The product was TLC pure. Overall yield was 892 mg (31%).

### Example 6

This Example sets forth a protocol for synthesizing 7-EMCMP.

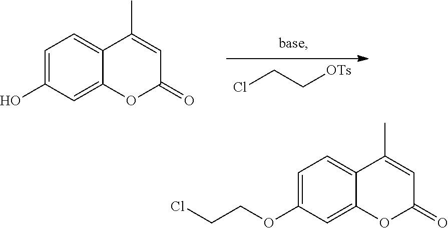 [SMILES: Cc1cc(=O)oc2cc(O)ccc12 ; Cc1ccc(S(=O)(=O)OCCCl)cc1 ; Cc1cc(=O)oc2cc(OCCCl)ccc12]

To a 500 mL two neck round bottom flask is added 4-Methylumbelliferone (17.61 g, 100 mmol) and 25% MeONa in MeOH (24.00 mL, 105 mmol). The mixture is stirred at room temperature to yield a yellow solution. To this solution is added DMF (100 mL) followed by 2-Chloroethyl p-toluenesulfonate (19.1 mL, 106 mmol) under stirring. Purging nitrogen flow is introduced from one neck to another. The mixture is slowly heated to 110° C. over 2 h, and allowed to react at 110° C. for another 4 h. The mixture is allowed to cooled to <70° C. and EtOH (200 mL) is added, then heated to boiling under gentle stirring for 10 min. The mixture is then cooled to room temperature, solids collected by vacuum filtration and rinsed with iced EtOH. The product is TLC pure (22.8 g, 96%).

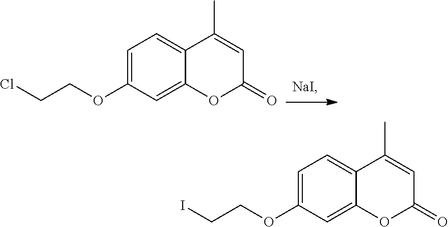 [SMILES: Cc1cc(=O)oc2cc(OCCCl)ccc12 ; Cc1cc(=O)oc2cc(OCCI)ccc12]

To a round bottom flask is added the product from the previous step (2.38 g, 10 mmol), NaI (3.00 g, 20 mmol) and acetone (25 mL). The mixture is fluxed overnight. The mixture is cooled to room temperature, followed by extraction between dichloromethane and water. The product (3.10 g, 94%) is found pure by HPLC fluorescence.

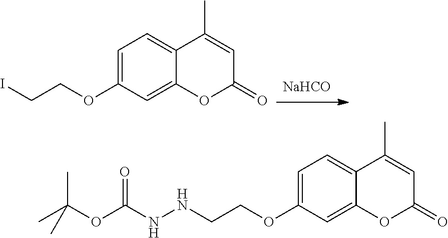

To a round bottom flask is added the product from the previous step (3.26 g, 10 mmol), t-butyl carbazate (1.58 g, 12 mmol), sodium bicarbonate (1.26 g, 15 mmol) and THF (10 mL). The mixture is stirred and refluxed at 65° C. for 16 hours (monitored by TLC, Ethyl Acetate:Hexane=1:1) The mixture is gravity filtered. The solution is optionally rotavapped to remove at least a portion of solvent, then extracted between dichloromethane (40 mL) and H₂O (20 mL×2). The organic layer is dried over MgSO₄ or Na₂SO₄, gravity filtered. The solvent is then removed by rotavap.

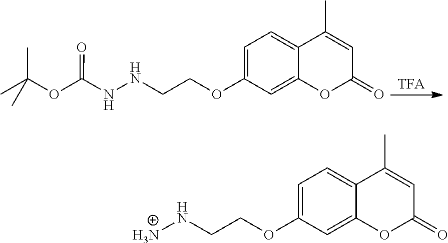 [SMILES: Cc1cc(=O)oc2cc(OCCNNC(=O)OC(C)(C)C)ccc12 ; Cc1cc(=O)oc2cc(OCCN[NH3+])ccc12 ; "TFA"]

To the round bottom flask containing the crude solid mixture from the previous step (without further purification) is added trifluoroacetic acid (TFA, 10 mL, 131 mmol). The solution is stirred for 1 h at r.t. (monitored by TLC, Ethyl Acetate:Hexane=1:1). The reaction mixture is used in the next step directly (“one-pot”).

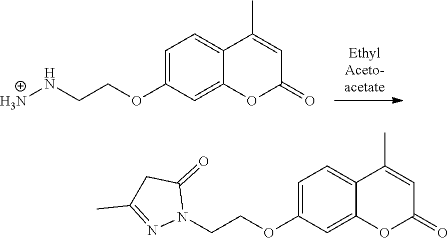 [SMILES: Cc1cc(=O)oc2cc(OCCN[NH3+])ccc12 ; CC1=NN(CCOc2ccc3c(C)cc(=O)oc3c2)C(=O)C1]

To the mixture from the previous step, ethyl acetoacetate (1.54 mL, 12 mmol) is added. The mixture is stirred for 3-16 h at r.t. (monitored by TLC, Ethyl Acetate, reactant Rf˜0, desired product Rf˜0.5, major side product Rf˜0.9). The mixture is added into a solution of Na₂CO₃ (5.30 g, neutralizing 100 mmol H+) in H₂O (40 mL). The solution is pH adjusted to 3-7 with any combination among sodium acetate, Na₂CO₃, NaHCO₃, NH₄Cl, NaOH, HCl or TFA. The mixture is extracted with dichloromethane (30 mL×4). The organic layer is dried over MgSO₄ or Na₂SO₄, gravity filtered. The solvent is then removed by rotavap.

The crude product is yellow to orange in color, as a combination of solid and very viscous liquid. The crude product is triturated with ethyl acetate (4.5 mL) at or near its boiling temperature, followed by dropwise addition of diethyl ether (5 mL) to precipitate the majority of the desired product.

### Example 7

This Example reports the results of a study conducted to compare the MS signal of an exemplar pyrazolone core-linker-coumarin core label, 4-MM, to that of PMP. As shown in FIG. 2, the MS signals of the two labels are similar.

### Example 8

This Example reports the results of a study conducted to compare the MS signal of an exemplar O-glycan, 3′-sialyllactose (“3′-SL”) after it was labeled with an exemplar pyrazolone core-linker-coumarin core label, 4-MM, to that of the same amount of 3′-SL after it had been labeled with PMP under the same conditions.

As shown in FIG. 3, the MS signal of 3′-SL labeled with 4-MM was lower than that of the same glycan labeled with PMP, but, importantly, the signal was of the same order of magnitude, indicating that O-glycans that can be detected by MS when they are labeled with PMP will also be detected when they are labeled with 4-MM.

### Example 9

This Example reports the results of a study comparing the pKₐ of PMP to that of an exemplar pyrazolone core-linker-coumarin core label, 4-MM.

A study of the pKₐ of the two labels indicated that the pKₐ of PMP is ˜7.86, while that of 4-MM is ˜7.61, or a difference of ˜0.25. It is believed that the modest difference in labeling efficiency between the two labels is because 4-MM is slightly more acidic than PMP.

### Example 10

This Example reports the results of a study comparing the labeling of an exemplar O-glycan with PMP or with an exemplar pyrazolone core-linker-coumarin core label, 4-MM, in a wholly aqueous solution to labeling the same glycan with the same labels under the same conditions, but in a solution 50% aqueous solution and 50% an exemplar polar, protic organic solvent.

The results of a study in which an exemplar O-glycan, 3′-SL, was labeled with PMP or with 4-MM, in either water or in a 50% solution of water and an exemplar polar protic solvent, methanol, are shown in FIG. 4. As can be seen in FIG. 4, the presence of the organic solvent did not significantly affect the labeling of the O-glycan with either label when the organic solvent constituted 50% of the solution.

### Example 11

This Example reports the results of a study conducted to determine whether an exemplar O-glycan, 3′-SL, labeled with an exemplar pyrazolone core-linker-coumarin core label, 4-MM, could be detected by UV, fluorescence, and MS.

A study conducted to determine whether an exemplar O-glycan, 3′-SL, labeled with 4-MM could be detected by UV, fluorescence, and MS. The results are shown in FIG. 5. As shown in FIG. 5, the labeled O-glycan could be detected by UV (the trace labeled “Absorbance Trace”), by fluorescence (the trace labeled “Fluorescence Trace”), and by MS (the trace labeled “Extracted Ion Chromatogram”). It is noted that the MS trace in FIG. 5 is offset from the other two traces because of the physical characteristics of the instrumentation used to introduce the sample into the mass spectrometer.

### Example 12

This Example reports the results of a study conducted to determine whether the linked D-glycose polymers present in maltodextrin (which are considered non-glycoprotein 0-glycans by structure) labeled with an exemplar pyrazolone core-linker-coumarin core label, 4-MM, could be detected by fluorescence and by MS.

A study conducted using 4-MM to label polymers of maltodextrin, which is made of a mixture of chains of D-glucose units of variable length, showed that the “ladder” of polymerized glucose units of maltodextrin labeled with 4-MM could be detected both by fluorescence and by MS. FIG. 6 shows the fluorescence trace and MS chromatogram for the first three degrees of polymerization (“DPs”) of maltodextrin of seven DPs detected by the study (for the purpose of presentation, the other four DPs were omitted from FIG. 6). As noted with respect to FIG. 5, the MS traces of the DPs in FIG. 6 are offset from the fluorescence trace of the same DPs because of the physical characteristics of the instrumentation used to introduce the sample into the mass spectrometer.

### Example 13

This Example reports the results of a study conducted to determine that the exemplar pyrazolone core-linker-coumarin core label, 4-MM, would label glycans known to be present in bovine fetuin, and whether it allowed the glycans to be detected both when they were present in the sample in a relatively high amount and in a relatively low amount.

A study was conducted testing the ability of 4-MM to label glycans known to be present in bovine fetuin, using samples in which the amount of bovine fetuin present differed by an order of magnitude (400 micrograms and 40 micrograms). The results are shown in FIG. 7. As set forth in FIG. 7, the glycans in the fetuin were released, labeled, and detected by MS at both concentrations of 4-MM. Other studies with 4-MM confirmed that it could be used to label and detect exemplar O-glycans using samples with 40 micrograms of glycan or glycans.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A fluorescent O-glycan labeling compound, said compound comprising a pyrazolone core, a linker, and, a coumarin core,
wherein,
(a) said linker covalently links said pyrazolone core to said coumarin core, and
(b) when said pyrazolone core is reacted with an O-glycan to label said O-glycan, thereby forming an O-glycan labeled with a derivative of said O-glycan labeling compound, said O-glycan labeled with said derivative of said O-glycan labeling compound is fluorescent,
wherein said compound has the structure of:
Structure 3, 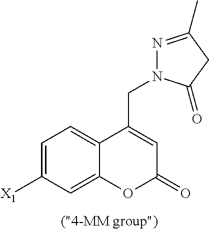
wherein X₁=Cl, Br, I, OH, OMe, OEt, NH₂, NMe₂, NEt₂,
Structure 4 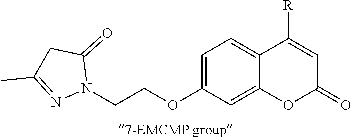
wherein R=CH₃ or CF₃,
of Structure 5, 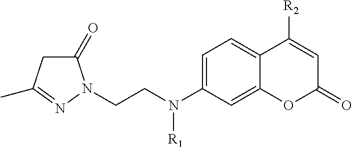
wherein R₁=H or CₙH₂ₙ₊₁; and R₂=CH₃ or CF₃,
Structure 6, 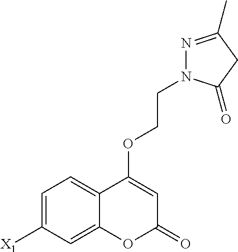
wherein X₁=H, Cl, Br, I, OH, OMe, OEt, NH₂, NMe₂, NEt₂,
Structure 7, 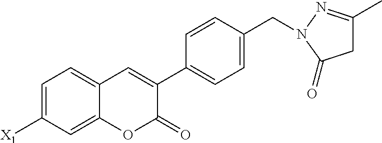
wherein X₁=H, Cl, Br, I, OH, OMe, OEt, NH₂, NMe₂, NEt₂, or
Structure 8, 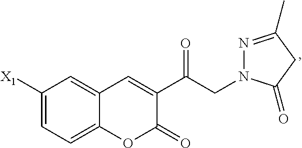
wherein X₁=H, Cl, Br or I.

2-11. (canceled)

12. The fluorescent O-glycan labeling compound of claim 1, wherein said compound is a compound of Structure 3, and has the structure of Structure 1:

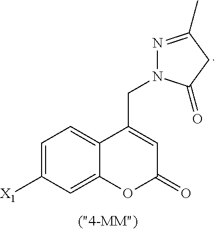

13. The fluorescent O-glycan labeling compound of claim 1, wherein said compound is a compound of Structure 4, and has the structure: 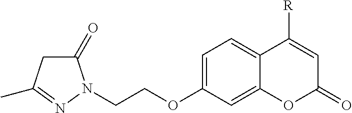
wherein R=CH₃ (“7-EMCMP”).

14. A method for releasing, labeling, and, optionally, analyzing, O-glycans present on a selected glycoconjugate of interest, said method comprising: incubating in a container said selected glycoconjugate of interest in a solution comprising a fluorescent O-glycan labeling compound, said fluorescent O-glycan labeling compound comprising a pyrazolone core, a linker, and, a coumarin core,
wherein
(a) said linker covalently links said pyrazolone core to said coumarin core, and
(b) when said pyrazolone core is reacted with an O-glycan to label said O-glycan, thereby forming an O-glycan labeled with a derivative of said O-glycan labeling compound, said O-glycan labeled with said derivative of said O-glycan labeling compound is detectable by fluorescence,
at a temperature of 0° C. to 100° C. for a time between about 10 minutes and 23 hours,
wherein said fluorescent O-glycan labeling compound has the structure:
of Structure 3, 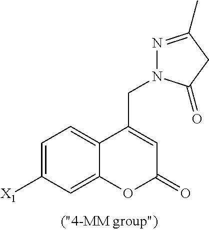
wherein X₁=Cl, Br, I, OH, OMe, OEt, NH₂, NMe₂, NEt₂,
of Structure 4 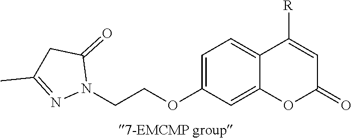
wherein R=CH₃ or CF₃,
of Structure 5, 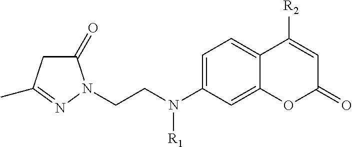
wherein R₁=H or CₙH₂ₙ₊₁; R₂=CH₃ or CF₃,
of Structure 6, 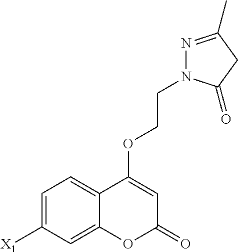
wherein X₁=H, Cl, Br, I, OH, OMe, OEt, NH₂, NMe₂, NEt₂,
of Structure 7 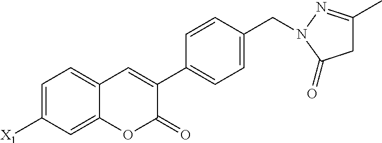
wherein X₁=H, Cl, Br, I, OH, OMe, OEt, NH₂, NMe₂, NEt₂, or
of Structure 8, 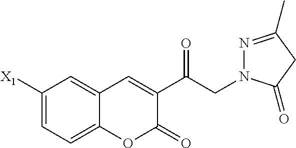
X₁=H, Cl, Br or I.

15-16. (canceled)

17. The method of claim 14, wherein said fluorescent O-glycan labeling compound is a compound of Structure 3, and has the structure:

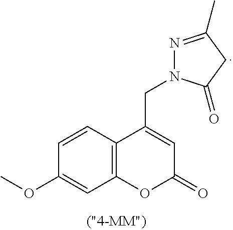 [SMILES: COc1ccc2c(CN3N=C(C)CC3=O)cc(=O)oc2c1]

18. The method of claim 14, wherein said fluorescent O-glycan labeling compound is a compound of Structure 4, and has the structure: 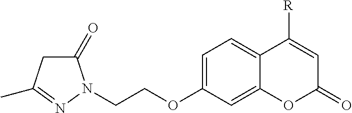
wherein R=CH₃ (“7-EMCMP”).

19. The method of claim 14, wherein said solution is wholly aqueous.

20. The method of claim 14, wherein said solution is a mixture of aqueous solution and an organic solvent that is either a polar protic organic solvent or an aprotic organic solvent.

21-23. (canceled)

24. The method of claim 14, wherein said solution further comprises an effective amount of a base.

25-26. (canceled)

27. The method of claim 24 wherein said base is dimethylamine, triethylamine (“TEA”), sodium hydroxide, potassium hydroxide, lithium hydroxide, or ammonium hydroxide, or a combination of two or more of these.

28-29. (canceled)

30. The method of claim 27, wherein said base is TEA.

31-40. (canceled)

41. The method of claim 14, wherein said selected glycoconjugate of interest is a glycoprotein or a glycopeptide.

42. (canceled)

43. The method of claim 14, wherein said aqueous solution contains no hydrazine.

44-47. (canceled)

48. The method of claim 14, further comprising analyzing said released, labeled O-glycans.

49-51. (canceled)

52. The method of claim 48, wherein said analysis comprises separating said released, labeled O-glycans by liquid chromatography and analyzing said released, labeled, and separated O-glycans by fluorescence, ultraviolet light, mass spectrometry, or a combination of two or more of these.

53-107. (canceled)

108. A kit for releasing and labeling O-glycans present on a glycoconjugate of interest, said kit comprising:
(a) one or more containers,
(b) a base selected from dimethylamine, triethylamine (“TEA”), sodium hydroxide, potassium hydroxide, lithium hydroxide, and ammonium hydroxide, or a combination of two or more of these, and
(c) a fluorescent O-glycan labeling compound, said compound comprising a pyrazolone core, a linker, and, a coumarin core,
wherein
(1) said linker covalently links said pyrazolone core to said coumarin core, and
(2) when said pyrazolone core is reacted with an O-glycan to label said O-glycan, thereby forming an O-glycan labeled with a derivative of said O-glycan labeling compound, said O-glycan labeled with said derivative of said O-glycan labeling compound is detectable by fluorescence, and further wherein said fluorescent O-glycan labeling compound is a compound of:
Structure 3, 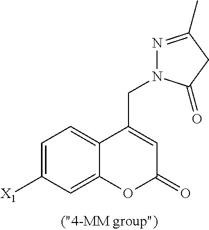
wherein X₁=Cl, Br, I, OH, OMe, OEt, NH₂, NMe₂, NEt₂,
Structure 4 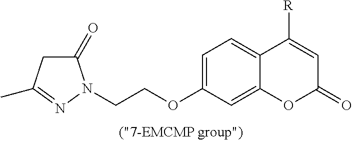
wherein R=CH₃ or CF₃,
Structure 5, 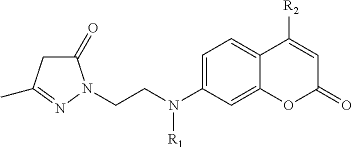
wherein R₁=H or CₙH₂ₙ₊₁; R₂=CH₃ or CF₃,
Structure 6, 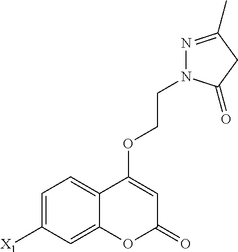
wherein X₁=H, Cl, Br, I, OH, OMe, OEt, NH₂, NMe₂, NEt₂,
Structure 7 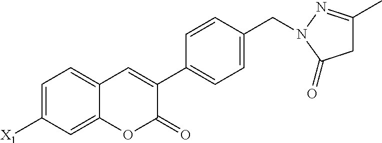
wherein X₁=H, Cl, Br, I, OH, OMe, OEt, NH₂, NMe₂, NEt₂, or
Structure 8, 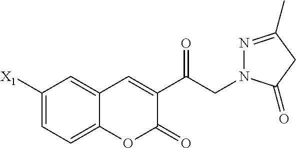
wherein X₁=H, Cl, Br or I.

109. (canceled)

110. The kit of claim 108, wherein said fluorescent O-glycan labeling compound is a compound of Structure 3, and has the structure of Structure 1:

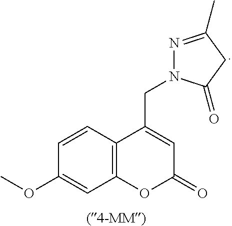 [SMILES: COc1ccc2c(CN3N=C(C)CC3=O)cc(=O)oc2c1]

111. The kit of claim 108, wherein said fluorescent O-glycan labeling compound is a compound of Structure 4, and has the structure: 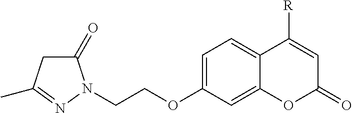
wherein R=CH₃ (“7-EMCMP”).

112. The kit of claim 108, wherein said base is TEA.

113. (canceled)

114. The kit of claim 108, further comprising: (d) at least one O-glycan standard.

115-116. (canceled)
